(19) United States
(12) Patent Application Publication
(10) Pub. No.: US 2014/0017337 A1
(43) Pub. Date: Jan. 16, 2014

(54) THERAPEUTIC METHODS

(71) Applicant: Paul Amoruso, Washington, DC (US)

(72) Inventor: Paul Amoruso, Washington, DC (US)

(21) Appl. No.: 13/546,202

(22) Filed: Jul. 11, 2012

(51) Int. Cl. A61K 36/53; A61K 36/8962; A61P 39/00; A61K 33/30

(52) U.S. Cl. 424/643; 424/745; 424/754

(57) ABSTRACT

Therapeutic methods includes several stages, including a modified fasting and low calorie stages, with low impact, moderate exercise, a sliming stage, and a maintenance stage. Nutritional therapeutics are also consumed during each phase, which support a plurality of metabolic and physiological functions.

### BACKGROUND

1. Field of Endeavor

The present invention relates to systems and processes useful as therapeutic methods, and more specifically to methods which can alleviate one or more conditions in a human.

2. Brief Description of the Related Art

Introduction

The vast majority of commercial diet programs take a simple, one-dimensional approach to weight loss. Some use basic calorie restriction without regard to the type or quality of food consumed, others rely on specific proportions of macronutrient (fat, protein, carbohydrate) intake, and still others use supplementation or meal replacement as a potential means of stimulating weight loss. Ultimately, these commercial diets are not designed from a medical perspective and do not address the full range of underlying issues driving weight gain and long term weight maintenance such as toxicity, metabolic function, diet composition, caloric intake and behavioral management. Most importantly, these diets are offered broadly to consumers independent of their health conditions, and therefore without an understanding of appropriateness or likely efficacy. As such, even when achieved, weight loss on these programs can take a long time to accomplish, and can be short-lived or unsustainable.

Program Categories

Dietary programs for weight loss can generally be divided into three main categories (some programs may fall into more than one category).

Calorie Restriction: These programs generally do not limit the types of food that you can eat, but focus primarily on the amount of food consumed. This is based on the assumption that energy in =energy out, and that to lose weight the only change one needs to make is to take in fewer calories than one burns in a day, with the ensuing caloric deficit leading to weight loss.

These diets may be termed MCD (modified calorie diet), typically meaning 1,200 or more calories per day, LCD (low calorie diet), generally indicating 800 to 1,200 calories per day, or VLCD (very low calorie diet), denoting up to 800 calories per day. In general, most experts agree that VLCD programs should only be performed under the supervision of a medical professional, as improper food intake can lead to micronutrient deficiencies.

Just about any diet (including those in other categories) can be turned into an MCD, LCD or VLCD simply by limiting the portion size of the allowed foods. Some examples of well known programs that primarily follow the tenets of calorie restriction are Jenny Craig, Weight Watchers and Nutrisystem.

Calorie restricted diets that ignore macronutrient content will fail to address the impact of different nutrient types on metabolism or on the nutritional needs of the body during weight loss and beyond. Often this results in loss of lean mass instead of fat. Further, such programs lack the hunger management tools that enable calorie restriction for meaningful weight loss in a reasonable period of time.

Macronutrient Modification: These programs do not necessarily limit the amount of calories that one consumes, but instead dictate parameters for the percentages of the different types of macronutrients that one can have as a component of their total caloric intake. Some examples include the USDA Food Pyramid, Zone Diet, Atkins, South Beach, Sugar Busters and Optimal Diet.

While there are in fact macronutrient ratios to which one should adhere, such ratios can vary based on the objective of the diet program (weight loss versus weight maintenance). These diets often get the ratios wrong or have no scientific support for their particular program structure. Further, these programs frequently ignore the quality of macronutrient (e.g., type of fat, organic versus non-organic), and as such can have a negative health impact including long term weight gain.

Supplementation Driven: Within this category are diet plans that add supplements to a food plan and meal replacement systems that use a shake, single food type, bar, etc. to replace a defined number of meals or portions each day. Most of the meal replacement plans allow at least one real, food-based “sensible” meal per day, and the vast majority of plans also fall into the Caloric Restriction category as well. Some examples of supplement driven diets include SlimFast, MediFast, MasterCleanse, B-12 Injection Programs, Dexatrim, Hydoxycut, Cookie Diet, Grapefruit Diet, Acai Berry Diet and Alli.

Diets that do not create sustainable eating habits or provide education related to nutrition that can guide future food choice are destined to fail or at least fail to result in sustainable weight loss. Further, many supplements marketed for weight loss have little or no real biological impact (meaning a placebo effect, if any), are fad diets with no scientific foundation or sacrifice long-term health for short-term impact. As such, medically selected and designed supplementation is critical to proper integration of these components into a diet protocol.

There remains a need, unmet by prior programs, for therapeutic methods, including those which can effectively reduce a participant's weight.

### SUMMARY

According to a first aspect of the invention, a therapeutic method for a human participant comprises (1) detoxifying the participant while fasting, (2) after (1), consuming by the participant a low-calorie diet, (3) after (2), consuming by the participant a medium-calorie diet, and (4) after (3), consuming by the participant a diet with a higher calorie content than the medium-calorie diet.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a comparison of the common causes of diet failures with solutions according to principles of the present invention;

FIG. 2 illustrates a weight loss program in accordance with a first exemplary embodiment of the present invention; and

FIGS. 3A and 3B illustrate tables comparing features of exemplary embodiments of programs of the present invention, with those of commercially available programs, in which the numbered columns represent the following: 1 screening; 2 fast results; 3 organic food; 4 behavior change; 5 sustain loss post diet; 6 nutrition education; 7 metabolism reset; 8 hunger support; 9 appetite reset; 10 anti-inflammatory; 11 scientific basis; 12 nutrient balance; 13 glycemic balance; 14 cleanses toxins; 15 stimulant-free; and 16 safe for digestion.

### DETAILED DESCRIPTION OF EXEMPLARY EMBODIMENTS

Referring to the drawing figures, like reference numerals designate identical or corresponding elements throughout the several figures. Some aspects of the present invention are described in the WEIGHTNOT “participant handbook”, available from WeightNot, Inc. (ca. Jul. 15, 2011; Vienna, Va.), the entireties of which are incorporated by reference herein.

A weight loss program is, by definition, a short term program or health intervention that enables someone to achieve a healthy body weight and body composition by aiding in overcoming the body's many levels of resistance to such physiological change. Since the drop-out rate for any program involving caloric restriction skyrockets beyond the period of just 60 days, success should be measured primarily by the level and rate of safe weight loss achievement—specifically, the loss of fat and NOT lean mass. Once someone has reached their target weight, long term maintenance of body composition is dependent on a healthy lifestyle, including good eating habits and exercise. While one's lifestyle after completing a weight loss program is largely independent of the short term program itself, ideally the program becomes the gateway to healthy living and facilitates changes in behavior that allow for sustained weight loss.

As such, a secondary measure of program success is the extent to which the program sets you on a path for improved health and creates a window of opportunity for adopting or returning to positive habits—though ultimately, the lifestyle choices following a program are the personal responsibility of the program participant.

Programs embodying principles of the present invention are designed to maximize your chance of weight loss success by screening to ensure that the diet approach is right for you from the outset, and by using an intensive but sustainable short term regimen that addresses hunger and supports the body's weight loss process. Programs embodying principles of the present invention include tracking tools which provide ongoing measurement of progress, so that along with staff support, a participant will be able to carefully monitor advancement toward diet goals. Finally, programs embodying principles of the present invention can restore and rebalance many key physiological processes, and provides assistance for the critical transition back to more normal eating patterns, helping to maximize the chances of weight loss maintenance following the program.

Programs embodying principles of the present invention incorporate a medical perspective across the entire program to maximize weight loss and safety for the participant.
Screening: Medical contraindications to programs embodying principles of the present invention are detailed to help determine if the program is safe.
Medically Designed Program: A program developed and optimized through extensive research and development, and through administration to thousands of participants under medical oversight.

Nutritional Therapeutics
Pharmaceutical Grade Therapies: The use of a high quality, therapeutic regimen(s) to restore metabolic and digestive function, and to address systemic issues such as inflammation and allergies.
Phone Support: Response to issues or concerns that arise during the course of the program.

Nutritional Therapeutics as described herein can be taken by or administered to a participant in a program embodying principles of the present invention to achieve one or more of numerous therapeutic objectives. Thus, it is the effect to be achieved which is a focus of the therapy, which can be achieved through the use of one of numerous methodologies. As described in greater detail elsewhere herein, specific examples of methodologies are presented, but functional equivalents can be employed, as summarized in Table 1 below.

TABLE 1
Therapeutic Objectives and Representative Methodology(ies)

| Therapeutic Objective* | Representative Methodology(ies) |
|---|---|
| Liver and Gall Bladder | Such as through the use of nutrients supporting bile production, fat |
| Support | transportation and liver cell protection |
| Detoxification Support | Such as through the use of nutrients supporting stage I and stage II |
| | detoxification in the liver |
| Kidney Support | Such as through the use of nutrients shown to improve markers of |
| | kidney function (such as serum albumin and creatinine levels) and |
| | protect kidney tissue |
| Adrenal Stimulation | Such as through stimulant elimination and the use of nutritionals |
| Reduction | shown to calm adrenal gland and promote healthy HPTA |
| | (hypothalmus-pituitary-adrenal) axis function |
| Anti-Catabolic/Lean Mass | Such as through the use of nutrients aiding in the reduction and |
| Protection | stabilization of Cortisol |
| Systemic Anti- | Such as through the use of antioxidants, essential fatty acids (for |
| inflammatory Therapy | example, EPA & DHA), natural COX-2 inhibitors, and through |
| (including Cardiovascular | the elimation of dietary allergens, irritants, chemicals, toxins |
| and Digestive) | |
| Reduction of Retained | Such as through the reduction of excess sodium and Cortisol |
| Water | |
| Broad Sprectrum Nutrient | Such as through therapeutic dosing of broad spectrum multi- |
| Restoration | vitamins |
| Enhanced Restorative or | Such as through fasting period(s) and sleep quality improvement |
| Healing Response | to maximize endogenous HGH production |
| Hormonal Balancing | Such as through the elimination of estrogenic substances (e.g. |
| | phyto-estrogens) and the use of nutritional precurors to hormones |
| | such as estrogen, testosterone, dhea, pregneolone, melatonin |
| Good Cholesterol (HDL) | Such as through healthy dietary fatty acid consumption and |
| Support | nutritional supplementation such as niacin |
| Bad Cholesterol (LDL) | Such as through reduction in unhealthy dietary fats, inflammation, |
| Reduction | and use of nutritionals such as lycopene and plant sterols |
| Sleep Support | Such as through the use of herbal or hormonal nutrients including |
| | GABA, inositol and melatonin |
| Anti-Excitatory/Brain | Such as through the elimination of chemicals and stimulants, the |
| Calming | use of nutrients to minimize agitation, stress response or damage |
| | from stress response, and stimulation of endogenous oxytocin |
| Adrenal Recovery (from | Such as through the replenishment of catecholamines using |
| fatigue/overstimulation/ | folates, glandulars or adaptogenic herbs |
| burnout) | |
| Mood Support | Such as through the use of serotonin precurors or nutrients that |
| | support neurotransmitter production |
| Energy Support | Such as through the use of nutritionals for thyroid hormone |
| | precursors/endocrine support, medium chain triglycerides (for |
| | immediate energy) and B vitamins |
| Circulating Fat Reduction | Such as through the reduction in select dietary fats that drive |
| | triglyceride and other serum markers |
| Blood Pressure Reduction | Such as through sodium reduction, vitamin D and potassium |
| | supplementation and natural calcium channel blockers, ACE |
| | inhibitors and beta blockers |
| Mitochondrial Function | Such as through the use of nutrients supporting cellular/ATP |
| Support | energy production like carnitine, CoQ10, creatine, EGCg, |
| | resveratrol and Krebs cycle intermediates |
| Electrolyte | Such as through the use of electrolyte/sodium supplementation |
| Maintenance/Replenishment | |
| Anti-Aging | Such as through the use of anti-oxidants, cellular protectants and |
| | SirT1 gene activators such as resveratrol |
| Brain Chemical Based | Such as through the use of serotonin precursors such as |
| Cravings Reduction | L-Tyrosine and 5-HTP |
| Yeast Overgrowth Based | Such as through the use of antimicrobials for candida yeast |
| Cravings Reduction | reduction and gut bacteria balancing |
| Blood Sugar and Insulin | Such as through the use of blood sugar stabilizing nutrients such |
| Management for Hunger & | as chromium, lipoic acid, biotin and taurine, anti-glycating |
| Craving Control | nutrients, dietary macronutrient combinations and low glycemic |
| | foods |
| Improved Circulation | Such as through the use of natural blood flow enhancers such as |
| | gingko, ginseng and precursors to nitric oxide |
| Bowel/Colon Support | Such as through the use of fiber supplementation |
| Improved Digestion and | Such as through the use of mineral chelates and digestive enzymes |
| Nutrient Absorption | such as protease, amylase and lipase, and the use of probiotics |
| Brown Fat Activation | Such as through the use of GLA (omega-6) |
| Fat Burning Support | Such as through the use of CLA (fatty acid) |
| Restored Bacterial Balance | Such as through the use of anti-microbials and probiotics |
| Gut Lining Restoration/ | Such as through the use of nutrients such as L-glutamine, |
| Mucosal Healing | glucosamine and citrus pectin |
| Fat Storage Signaling | Such as through the reduction of insulin, cortisol and estrogenic |
| Reduction | nutrients |
| Fat-Generated Estrogen | Such as through the reduction of adipose tissue volume |
| Reduction | |
| Hunger Management | Such as through improved leptin sensitivity and stimulation of |
| | endogenous CCK and galanin |
| Macronutrient Balancing | Such as through prescribed dietary combinations to achieve and |
| for Body Composition | maintain body composition ratios |
| Management | |
| Calorie Control for Body | Such as through prescribed dietary portions by food type to |
| Composition Management | achieve and maintain body composition ratios |
| Taste Reset/Recalibration | Such as through the removal of chemicals, additivies, sodium, |
| | sweeteners from diet for an extended or indefinite period(s) |
| Ketosis for Adipose Tissue | Such as through the restriction of dietary carbohydrates and |
| Reduction | calorie intake to prompt burning of stored body fat for energy |
| Hydration | Such as through the intake of a prescribed volume of low or no- |
| | calorie fluids to facilitate therapies such as detoxification, and to |
| | maintain proper metabolic function |
| Carbohydrate Absorption | Such as through the use of supplementation such as green coffee |
| Management | bean extract, white bean extract and fiber |
| Hunger Signaling | Such as through the elimination of artificial sweeteners that send |
| Restoration | false hunger signals, and elimination or reductin of dietary |
| | chemicals and cortisol that blunt satiety signaling |
| Thyroid Nutrient | Such as through the elimination of chemicals that leach thyroid |
| Restoration | hormone precursors |
| Red Blood Cell | Such as through the use of nutrients that promote blood cell |
| Promotion/Anti Anemia | production such as vitamin B complex, vitamin C and vitamin E, |
| Support | and dietary sources of iron |
| Fat Availability | Such as through the increase of stored and circulating fat soluble |
| Improvement/Fat Burning | vitamins |
| Resistance Reduction | |
| Insulin Sensitivity | Such as through the reduction in insulin spikes and overal insulin |
| Improvement/Restoration | levels by dietary control and supplementation to address metabolic |
| | syndrome, pre-diabetes and Type II diabetes |
| Mental Acuity and | Such as through the use of nutrients such as inositol and |
| Memory Improvement | L-carnitine |
| Allergen Resistance/ | Such as through the use of nutrients such as quercetin and vitamin |
| Immunoresponse Improvement | C |

*Achieved through diet composition/protocols and nutritional supplementation

Nutrition Lipolysis Overview

Nutrition Lipolysis is a medically designed process using food and pharmaceutical grade therapies to cause the rapid breakdown and elimination of fat in the body. While other weight loss approaches may cause the loss of water weight or lean mass, the Nutrition Lipolysis process targets exclusively fat. Beyond the simple calorie or food restrictions of other weight loss programs, Nutrition Lipolysis involves the restoration and management of the body's metabolic functioning to facilitate and maximize fat loss and to help sustain weight loss aided by the a Nutritional Therapeutics regimen.

Multi-Stage Medically Designed Weight Loss Protocol

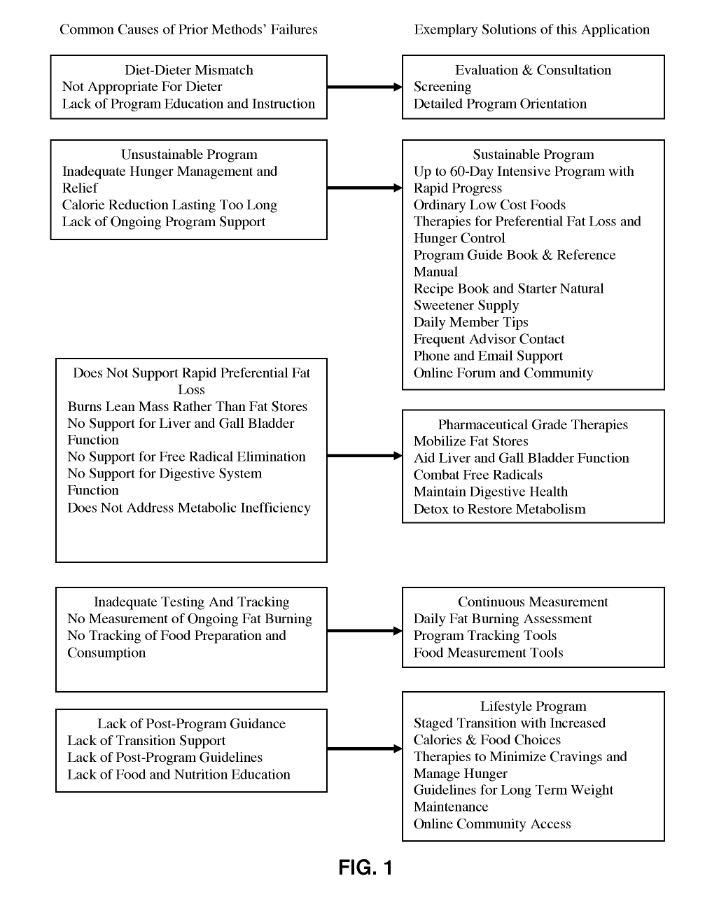

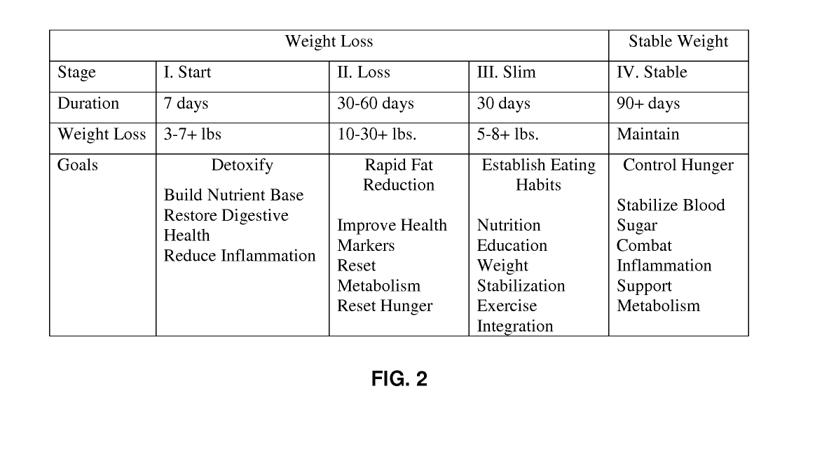

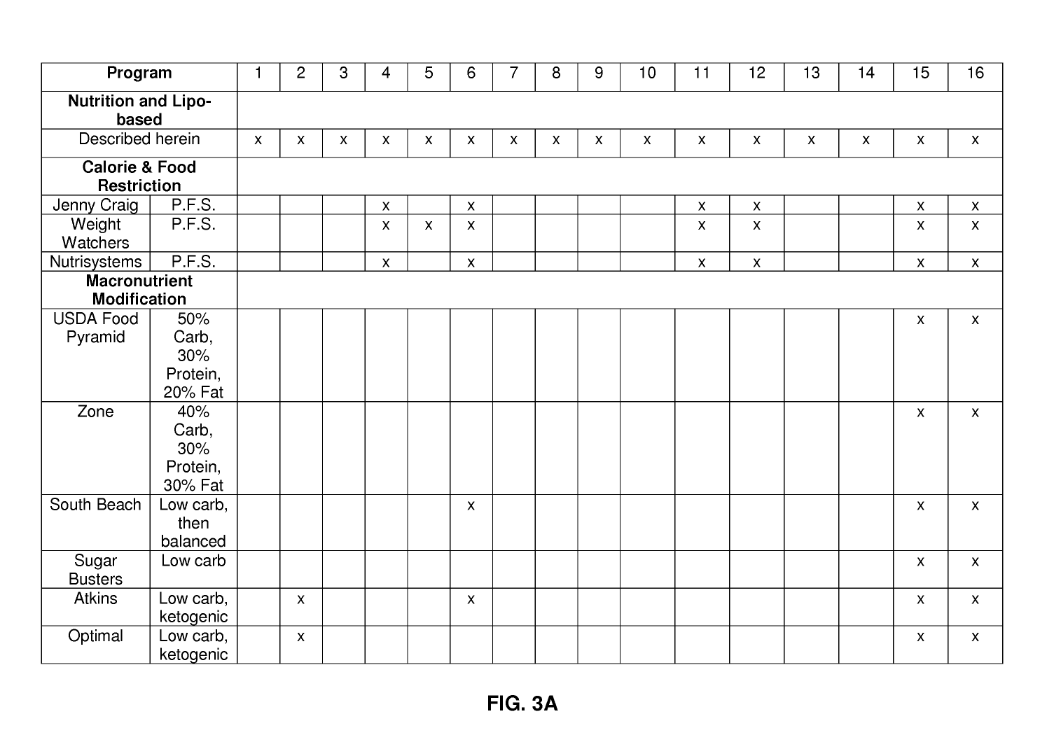

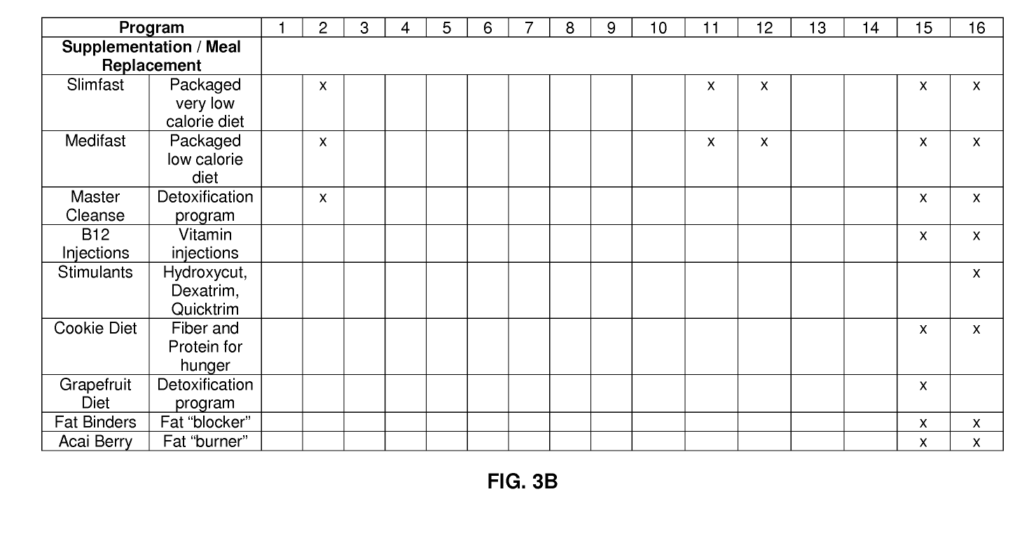

Based on the Nutrition Lipolysis principles, a multi-stage, medically designed weight loss protocol addresses the full range of underlying issues driving weight gain and long term weight maintenance—toxicity, metabolic function, diet composition, caloric intake, and behavioral management. multi-stage, medically designed weight loss protocol involves at least four distinct phases (see FIG. 1):
Start rids the body of toxins that contribute to weight gain and cause resistance to weight loss, reduces inflammation and allergic responses and prepares the body for rapid weight loss.
Loss safely and quickly eliminates excess fat while preserving lean mass, combats the hunger typically associated with low calorie diets and improves health markers such as blood pressure, good cholesterol and blood glucose.
Slim enables further weight loss with an increased caloric and macronutrient intake (MCD or modified calorie diet).
Stable Weight establishes eating patterns that allow for stabilization of weight long term.

The duration of the first three weight loss stages is up to 13 weeks, depending on the program selected. In combination, these three program phases optimize weight loss results and drive measurable health improvement, in addition to establishing the nutritional and behavioral foundation for long term weight management success.

Designed to Achieve the Diet Ideal

The primary mechanism for weight loss in any weight loss protocol is the creation of a structural deficiency in caloric intake versus calories burned by the body. In a state of calorie deficit, the body responds by burning available internal energy stores to provide needed nutrition and by adjusting its metabolic rate to slow calorie burning as an adaptive measure. From a behavioral standpoint, weight loss programs also serve as a means of interrupting current habits and establishing new patterns of food consumption. As such, the best programs will ensure that:

1) the internal energy source used by the body is unwanted fat and not lean mass,

2) the body is prepared and supported in its efforts to efficiently burn fat as fuel,

3) metabolism and calorie burning do not slow to the point that weight loss is minimized,

4) general nutrition during the program is maintained,

5) hunger from reduced caloric intake is managed and minimized,

6) the participant is engaged and behaviors are reinforced through rapid, measurable results, and

7) program guidelines lay a foundation for new long term eating habits.

The medically designed weight loss protocol was created to meet all of these criteria for an ideal program, supported by extensive education and counseling. Each of the phases word in combination to create an optimal weight loss result. In some cases, based on the initial screening for contraindications, all participants complete each of the stages in immediate succession to achieve the very best outcome.
Start prepares the body for rapid weight loss while also initiating fat reduction (3-7+ pounds)
Loss is an intensive phase of substantial fat loss (10-15+ pounds over 30 days, 16-30+ pounds over 60 days)
Slim locks in the healthy eating habits that ensure weight loss maintenance, stabilizes weight and typically results in further fat reduction (5-8+ pounds)
Stable Weight provides support for ongoing weight maintenance while reinforcing healthy habits.

Participating in all four stages of the protocol provides the best chance of success and is ideal for achieving the highest level of weight loss and weight maintenance post-program. If for reason of time constraints or some other consideration, a participant should be unable to participate in all stages, each completed program stage should still individually accomplish its goals. However, for those who opt to skip program stages:
there may be slightly less rapid weight loss without the Start phase
metabolism may not be restored as rapidly or as fully, meaning less efficient energy burning for some period without the Start phase
the participant may experience more cravings during Loss without the Start phase
the participant may experience more detoxification effects during Loss without the Start phase
there may be more fluctuation in participant weight following Loss without the Slim phase
there may not be additional weight loss following Loss without the Slim phase
the participant may return to previous eating habits following Loss without the Slim phase
there may be more fluctuation in weight following Slim without Stable Weight for ongoing weight maintenance.

Overview

Systems and programs embodying principles of the present invention can include a comprehensive daily regimen of nutritional supplements that have been scientifically designed and proven to support weight loss during each stage of the program. All of the nutritional therapeutics are advantageously:
physician grade
third-party tested
non-prescription
made without stimulants
made without artificial substances
free of common allergens
selected based on clinical studies demonstrating efficacy and safety
provided in therapeutic doses
safe to take with other prescription medications

Even with a well-designed, calorie restricted food plan, diets will commonly fail altogether or result in a reduction of lean mass instead of fat. This is because the body is most often nutritionally and metabolically unprepared for fat loss, and as such is resistant to fat burning—meaning that weight loss can be unpleasant and a struggle, with results that are slow and inadequate. Nutritional therapeutics embodying principles of the present invention are designed to address the most common nutritional and metabolic reasons for diet failure, and to ensure that you burn fat, not lean mass. A meal plan alone is NOT enough to achieve consistent weight loss success, which is why nutritional therapeutics in accordance with the present invention can be a critical component of every stage of a system embodying principles of the present invention.

Ten (10) Nutritional Therapeutics
Detoxification and Liver Support: Reducing fat-stored toxins so that the body does not resist fat burning, while aiding the efficient functioning of the liver for toxin and fat processing
Insulin and Glucose Stabilization: Maintaining consistent blood sugar and insulin levels to help avoid acute hunger and combat insulin resistance
Endocrine Support: Replenishing nutrients necessary for efficient thyroid functioning and hormone production
Carbohydrate Craving and Mood Support: Providing nutrients that support Serotonin production to minimize carbohydrate cravings
Digestive Health and Colon Support: Introducing fiber that maintains digestive regularity
Good Cholesterol and Anti-Inflammatory Support: Supplementing dietary fat with good cholesterol sources to minimize inflammation and restore healthy cholesterol ratios
Hunger Support: Causing the signaling of fullness to the brain to avoid acute hunger
Fat Mobilization Support: Establishing a sound base of fat soluble nutrients so that the body does not resist fat burning
Energy Support: Providing nutrients for improved cellular energy production and fat burning
Cortisol Stabilization: Mitigating the secretion of fat storage hormones in response to stress and inflammation

Ingredients

While nutritional therapeutics in accordance with the present invention involve a combinations of substances, all are natural and many are quite familiar—B vitamins, Zinc, Magnesium, Chromium, Ginseng, Omegas, fiber and numerous others. When administered in therapeutic doses and in the right combination with the right timing, these essential substances provide exactly what the body needs to accomplish rapid, healthy weight loss during a program as described herein.

START Phase

Toxin Exposure and Consumption
The average American unknowingly eats about 124 pounds of additives per year
According to the EPA, more than 22.5 billion pounds of toxic chemicals (PBT and Non-PBT) were released into the ground in the year 2008 alone
Each year over 2.5 billion pounds of pesticides are released onto crop lands, forests, and lawns

Detoxification (or detox) is the process by which the body neutralizes, breaks down and excretes the thousands of toxic substances with which we come into contact every day. The Start phase of a program embodying principles of the present invention is a preferably 7-day plan designed to restore and accelerate the detoxification process in your body to maximize your diet program results.

Toxin Categories

Unlike food and drugs, chemicals are not regulated by a government agency; therefore companies manufacture and sell chemicals for cleaning, refining, stripping, modifying growth and killing pests without regulatory oversight. Some estimates indicate that as many as 80,000 chemicals are in use without any idea of their long-term effects on health or the environment.

The primary classes of toxins include:

Biological
Macromolecules (Cholera toxin, Aflatoxin, Glycated collagen, Lipofuchsin)
Small molecules (Agent orange, Ammonia, Acetaminophen, Lactose)

Metals
As, Al, Cd, Hg, Pb, U, Zr, Ti, Sr, Ba, Bi, Zn, Cu, Cr, Mn

Xenobiotic
Macromolecules
GM[O] foods

Organotoxins—Low (<500 Amu) Molecular Weight
Solvents
Phthalates
Chlorinated pesticides
Polychlorinated biphenyls (PCBs)
Acetaldehyde—this proven carcinogen can be found in alcohol consumption, cigarette smoke, vehicle and factory exhaust, candida overgrowth, synthetic fragrances, drinking from polyethylene plastic bottles—even campfires

Absorbing Toxins and Health Impact

Toxin exposure occurs from sources inside and outside of the human body. Humans' own biological processes create toxins that we must neutralize and excrete. Additionally, we are exposed to toxins from pollution in the air, the ground, our water and our food. Our skin, lungs, and digestive tract are all points of entry for these external toxins. Mercury alone can be found in auto exhaust, paint and paint solvents, pesticides, amalgams, drinking water, plumbing and wood preservatives.

All toxins poison the body to some extent, causing our systems to function less efficiently, and decreasing our lifespan and quality of life through the development of diseases like Parkinson's, Fibromyalgia, Lupus, Diabetes, Obesity, and countless others. As an illustration, one study looked at the effect of the toxin acetaldehyde (from vehicle exhaust) on the “biological clock” of traffic officers vs. office workers by examining telomere length. Telomeres are protective caps on our chromosomes that naturally get shorter with age, but can also be worn down faster with toxic exposure and diseases. The study showed that traffic officers uniformly had more telomere shortening than office workers, and those officers in areas with greater air pollution had even more shortening than those in light traffic areas. Pollution really does kill

Detoxification Process

The body has incredible detoxification powers all on its own. However, it needs the proper fuel to carry out these processes, mostly in the form of proper nutrition. This is one of the major reasons that programs embodying principles of the present invention stress eating organic food as much as possible. Not only do organic foods have fewer toxins from pesticides, antibiotics, and hormones, but they also provide a much higher nutrient density compared with the same volume of non-organic food.

Most detoxification occurs in the liver in two distinct phases. In Phase I detoxification, the liver uses specific enzymes to change toxic substances into intermediate products. In Phase II, large water soluble molecules can be added to them so that they can then be safely excreted through the kidneys or the GI tract. Phase I detoxification requires Niacin, Magnesium, Copper, Zinc, Vitamin C, Vitamin B2, B3, B6, B12, folate, and flavanoids to operate properly. A deficiency in any one of these micronutrients can slow the detoxification process significantly.

Phase II detoxification requires a host of different amino acids and micronutrients to function properly. It is essential that these substances be present, because the intermediate products in Phase I can be as or even more toxic than the starting toxin, and as such these intermediate products must be eliminated quickly. Any deficiency of the nutrients required for this stage can lead to progressively worsening illness or obesity, as the body's only defense is to hold on to extra fat stores which serve to sequester the toxins that it can't eliminate.

In spite of the detoxification capabilities of our bodies, toxins do inevitably build up over time. This is a function of the level and long term nature of our exposure, and the variable nature of our (often poor) nutrition and exercise habits. As such, most people need extra help.

Programs Embodying Principles of the Present Invention

Programs embodying principles of the present invention include an accelerated detoxification and modified calorie phase that typically results in initial weight loss (2 to 5 pounds or more), as well as improving response to the low calorie phase by better enabling the human body to process fat and toxins released from stored fat. Pharmaceutical-grade supplements give the liver fuel for detoxification, and allow the body to clear some of the toxic “backlog” in a very short period of time. The programs are advantageous for individuals who wish to maximize the results of their weight loss program, with potential benefits including improved energy, digestion, reduced inflammation and allergies, and of course more efficient processing of body fat for energy. Because results may vary among individuals based upon that individual's starting toxic load, some participants may benefit from additional phases of detoxification following a low calorie phase of the program.

There can be a significant release of stored toxins in the participant's body as a result of following the program guidelines. As the body processes these toxins, it is possible that the participant may experience symptoms that reflect your body's efforts to eliminate these toxins, such as headache, fatigue, body aches, nausea, acne, and mood changes. Each person varies in their individual response, which is not predictable in advance. For most, these symptoms are mild when they do occur, and are easily managed. While the symptoms noted above may in some cases persist throughout the 7-day program and into the next phase of the program, more typically, program participants will see an increase in energy, improvement in mood, feeling of well-being, and weight loss of 3-7+ pounds.

### Example

In general terms, at day 1, the participant begins a 2-day modified fast, accompanied by nutritional therapeutics and walking. At day 3, for example, the participant begins a modified calorie diet, also accompanied by nutritional therapeutics and walking. At day 7, for example, the end of the modified calorie diet is reached, accompanied by nutritional therapeutics and walking. At day 8, for example, the next stage (described below) commences.

Days 1-2: Fasting with Nutritional Therapeutics

The participant does not eat any foods during days one and two; instead, the participant will be using only the nutritional therapeutics supplements (“NTx” hereafter) noted below. These two days are designed to minimize the ongoing toxic load from new sources to provide some immediate relief to the body, and to restore the nutrient base that enables detoxification in the body. Advantageously, the participant should also begin walking 10,000 steps per day (this will continue through the entire program).

Morning
2 Scoops of NT1 powder mixed with 8 ounces of water, pure fruit juice, rice milk or almond milk. (120 calories from rice milk/juice/almond milk maximum). Two scoops is equal to 4 level tablespoons or 12 level teaspoons, not heaping. For Days 1 and 2 only, if water is used to mix NT1, the participant may have a glass of juice, rice milk or almond milk separately (up to 120 calorie serving maximum). On Days 3-7, those who use water to mix NT1 should have the snack portion as specified, and NOT separate juice, rice milk or almond milk.
3 NT2 capsules

Evening (10-12 Hours after Morning Supplementation)
2 Scoops of NT1 powder mixed with 8 ounces of water, pure fruit juice, rice milk or almond milk (120 calories from rice milk/juice/almond milk maximum
3 NT2 capsules

Days 3-7: Modified Calorie Diet with Nutritional Therapeutics

During these five days, the participant will continue with a slightly expanded supplementation regimen, while eating a modified calorie diet.

Morning•2 Scoops of NT1 powder mixed with 8 ounces of water, pure fruit juice, rice milk or almond milk, as noted above.
3 NT2 capsules
2 NT3 capsules

Evening (10-12 Hours after Morning Supplementation)
2 Scoops of NT1 powder mixed with 8 ounces of water, pure fruit juice, rice milk or almond milk, as noted above.
3 NT2 capsules
2 NT3 capsules

Breakfast:

1. One serving of lean meat, fish, beans or eggs in a breakfast portion.

2. One serving of fruit in a breakfast portion.

Lunch:

1. One serving of lean meat, fish, beans or eggs, as noted above.

2. One serving of vegetables OR leaf greens, as noted above.

Optional Snack:

One ounce of nuts, or 3.5 ounces of avocado, or 4 ounces of olives (pitted, no filling, Kalamata olives not allowed), daily, as a substitution for juice, rice milk or almond milk when water is mixed with NT1 powder (both daily servings).

Dinner:

1. One serving of lean meat, fish, beans or eggs.

2. One serving of vegetables OR leaf greens.

3. One serving of fruit.

Seasoning: The juice of one (100 gram) lemon OR one (65 gram) lime daily is allowed (equivalent of 3 tablespoons). Fresh and dried herbs or spices without added sugar or starches are allowed.

Beverages: 2 liters of water should be consumed daily. The participant may drink herbal tea in any quantity without sugar. No milk, coffee, or alcohol is allowed. Stevia or Xylitol, which are natural sweeteners, may be used. Preferably, artificial sweeteners, like Saccharin, are avoided as much as possible, as the body does not process these well.

Measuring Food:

All food should be measured raw and prior to cooking. All visible fat and skin should be carefully removed before cooking.

Cooking: Food may be prepared (or dressed) with up to one tablespoon of extra virgin olive oil, flaxseed oil or sunflower oil per day. No butter or other oils are allowed.

Calories by Food Category (WOMEN & MEN)

| Food Category | Breakfast | Lunch | Dinner | Sub-total |
|---|---|---|---|---|
| Protein | 100 | 130 | 130 | 360 |
| Veg. or Greens | 0 | 30 | 30 | 60 |
| Fruits | 70 | 70 | 0 | 140 |
| Oil & Seasoning | 30 | 50 | 50 | 130 |
| NTx (w/o allowed Bev.) | 135 | 0 | 135 | 270 |
| Allowed Beverages | 120 | 0 | 120 | 240 |
| or allowed sub'n | | | | |
| Sub-total | 455 | 280 | 465 | |
| Total | | | | 1200 |

### Selecting Meats

As a source for lean protein, the participant may select white meat chicken, turkey, veal, lean beef (see below for specific beef selections) or game meat (see below for specific game meat selections). All portions are for raw, uncooked meat.

Meats Allowed—Category A:

Chicken (White meat), Kidney, Tripe, Turkey (White meat)

Breakfast Portion: 85 grams or 3 ounces Lunch/Dinner Portion: 115 grams or 4.05 ounces

Meats Allowed—Category B:

Beef <5% fat, Chicken (Dark meat), Duck, Game (Lean), Liver, Tongue (Veal only), Turkey (Dark meat),

Beef <10% fat, Game (Lean), Goose, Lamb (except ground), Pork (except ground) Turkey Sausage

Breakfast Portion: 75 grams or 2.65 ounces Lunch/Dinner Portion: 95 grams or 3.35 ounces

Meats Allowed—Category C:

Game (Fatty), Cured and Smoked Meats, including Bacon and Ham, Sausage—Beef/Pork/blood/other, Pate, Spam, Foie Gras, Lamb (ground), Pork (ground), Tongue (beef, lamb, pork)

Breakfast Portion: 60 grams or 2.1 ounces Lunch/Dinner Portion: 75 grams or 2.65-ounces

Nutritional Therapeutics

During the course of the Start phase of the program, the participant will take three dietary supplements to support your weight loss.
NT1: Two servings (2 scoops each) per day mixed with water, pure juice, rice milk or almond milk starting on day one. Includes N-Acetyl Cysteine, an immediate precursor to glutathione, a potent antioxidant and among the most important detoxification nutrients for the liver. Includes 5 g of fiber per serving. Fiber is essential to any cleansing protocol as it binds toxins and eliminates them through the GI tract. Includes nutrients for phase II liver detoxification. Includes a high ORAC vegetable extract blend and polyphenols.

Total Carbohydrates 18 g 6%

Dietary Fiber 5 g 20%

Soluble Fiber 2 g

Sugars 13 g

Protein 18 g 30%

Magnesium (as Citrate) 100 mg 25%

Potassium (as Citrate) 170 mg 5%

Oryzatein™ Whole Grain 18.75 g**

Brown Rice Protein Concentrate

Psyllium Husk Powder 4.16 g

Glycine USP 500 mg

Vegetable Anti-Oxidant 500 mg

Blend (2,500 ORAC) Containing:

Broccoli**

Broccoli Sprout Extract**

Tomato**

Spinach**

Kale**

Brussel Sprouts**

Onion Extract**

Glucosinolates 1,000 mcg**

Sulphoraphane Potential 500 mcg**

Bentonite 250 mg**

Taurine 250 mg**

L-Glutamine 150 mg**

Acetyl-L-Carnitine HCI 125 mg**

N-Acetyl-L-Cysreine USP 125 mg**

Green Tea Extract 50 mg**

(Standardized to contain 60% Polyphenols & 40% EGCG)

Alpha Lipoic Acid 50 mg**

Rosemary (aerial portion) 50 mg**

Schizandra Berry Extract 50 mg**

Ellagic Acid 25 mg**

Stevia Leaf Extract 5 mg**

*% Daily Values are based on a 2,000 calorie diet.

**% Daily Value not established
NT2: Three capsules twice per day starting on day one, each capsule containing (amount, % daily value):

Vitamin A 7,142 IU 143% (as Mixed Caroteniods from palm fruit tree)

Magnesium 200 mg 50% (TRACCS® Magnesium LYSYL Glycinate Chelate)

Magnesium 1 mg 50% (TRACCS® Magnesium Glycinate Chelate)

Chromium 200 mcg 167% (TRACCS® Chromium Nicotinate Glycinate Chelate)

Molybdenum 100 mcg 133% (TRACCS® Molybdenum Glycinate Chelate)

Potassium 100 mg 3% (as Potassium Glycinate Complex)

Vanadium 100 mcg*(TRACCS® Vanadium Nicotinate Glycinate Chelate)

Vitamin C (as Ascoribic Acid) 600 mg 1000%

Vitamin D (as Cholecaliciferol) 500 IU 125%

Vitamin B1 (as Thiamine HCI) 75 mg 5000%

Vitamin B2 (as Riboflavin) 75 mg 4411%

Vitamin B3 (as Niacinamide) 75 mg 375%

Vitamin B6 (as Pyridoxine HCI) 50 mg 2500%

Vitamin B12 (as Methylcobalamin) 500 mcg 8333%

Iodine (as Potassium Iodine) 200 mg 133%

Zinc (as TRACCS® Zinc Glycinate Chelate) 25 mg 167%

Selenium (as Selenium Glycinate Complex) 250 mcg 357%

Choline (as Citrate) 100 mg*

Inositol 100 mg*

Alpha Lipoic Acid 10 mg*

Trimethylglycine 200 mg*

Hesperidin 10 mg*

Quercertin DiHydrate 25 mg*

Rutin 25 mg*

PABA (Para-Aminobenzoic Acid) 25 mg*

Boron (as Boroganic glycine) 2 mg*

Biotin (as d-Biotin) 500 mcg 166%

Calcium (as TRACCS® Calcium Glycinate Chelate) 100 mg 10%

Pantothenic Acid (as d-calcium Pantothenate) 250 mg 2500%

Folates (NatureFolate™ Blend) 400 mcg 100%

Vitamin E (as Total Natural Tocopherols) 300 mg*

as d-gamma 205 mg*

as d-alpha 25 mg/37.5 IU 125%

as d-beta 10 mg*

as delta 60 mg*

Alpha Carotene*

*% Daily Value not established
NT3: Two capsules twice per day starting on day three. Three Capsules Contain (amount, % daily value):

Dandelion Root Extract 4.1 225 mg*

Choline Bitartrate USP 185 mg*

Inositol 140 mg*

L-Methionine USP 140 mg*

Milk Thistle Seed Extract 130 mg*(Standardized to contain 80% Silymarin)

Artichoke Leaf Extract 145 mg*(Standardized to contain 5% Cynarin) (from Asteracea Family)

Galic Bulb 100 mg*

Turmeric Root Extract 100 mg*(Standardized to contain 95% Curcumin)

Beet Leaf 65 mg*

*% Daily Value not established

The LOSS Stage

In general terms, for the first, e.g., two days the participant will eat a higher fat diet than will be followed throughout the program. This period of lipid loading will help to bolster the participant's normal fat reserves to help the participant not feel hungry during the low calorie portion of the program. It can be an important aspect for some participants to avoid alcohol during the phase of lipid loading and throughout a program in accordance with the present invention. On day 3, the participant will begin an, e.g., 820 calorie per day diet as directed. Small changes or “cheats” in the plan can decrease the participant's results, but are still within the spirit and scope of the present invention. This 820-calorie-per-day portion of the LOSS stage can last for many days, e.g., 30 or 60 days. At the fifth day of the LOSS stage, daily ketone measurements are advantageously made.

Days 1-2 Example

Breakfast

1. One serving of lean meat, fish or eggs from the lists described herein in the designated breakfast portion.

2. One serving of fruit from the lists described herein in the designated portion.

Lunch

1. One serving of lean meat, fish or eggs from the lists described herein in the designated lunch portion.

2. One serving of vegetables OR leaf greens from the lists described herein in the designated portion.

Snack: One 2.5 ounce serving of nuts, from the list described herein, or 8.75 ounces of Avocado, daily. The participant may have 4 ounces of Olives (pitted, not stuffed, Kalamata olives not allowed) as one half of a snack portion, in which case the participant could also have 4.25 ounces of Avocado or 1.25 ounces of Nuts

Dinner:

1. One serving of lean meat, fish or eggs from the lists described herein in the designated dinner portion.

2. One serving of vegetables OR leaf greens from the lists described herein in the designated portion.

3. One serving of fruit from the lists described herein in the designated portion.

Seasoning, beverages, measuring food, and cooking per above.

### Calories

| Food Category | Breakfast | Lunch | Dinner | Snack | Sub-total |
|---|---|---|---|---|---|
| Protein | 230 | 335 | 335 | 0 | 900 |
| Veg. or Greens | 0 | 60 | 60 | 0 | 120 |
| Fruits | 70 | 70 | 0 | 0 | 140 |
| Oil & Seasoning | 30 | 50 | 50 | 0 | 130 |
| NTx | 25 | 35 | 40 | 0 | 100 |
| Allowed Snacks | 120 | 0 | 120 | 4700 | 240 |
| Sub-total | 355 | 550 | 465 | 470 | |
| Total | | | | | 1860 |

Days 3—Completion

Breakfast:

1. One serving of lean meat, fish or eggs from the lists in the designated breakfast portion.

2. One serving of fruit from the lists in the designated portion.

Lunch:

1. One serving of lean meat, fish or eggs from the lists in the designated lunch portion.

2. One serving of vegetables OR leaf greens from the lists in the designated portion.

Dinner:

1. One serving of lean meat, fish or eggs from the lists in the designated dinner portion.

2. One serving of vegetables OR leaf greens from the lists in the designated portion.

3. One serving of fruit from the approved lists in the designated portion.

### Calories

| Food Category | Breakfast | Lunch | Dinner | Snack | Sub-total |
|---|---|---|---|---|---|
| Protein | 130 | 185 | 185 | 0 | 500 |
| Veg. or Greens | 0 | 30 | 30 | 0 | 60 |
| Fruits | 70 | 0 | 70 | 0 | 140 |
| Oil & Seasoning | 0 | 10 | 10 | 0 | 20 |
| NTx | 25 | 35 | 40 | 0 | 100 |
| Sub-total | 255 | 260 | 335 | 0 | |
| Total | | | | | 820 |

Pacing period dietary guidelines (days 3 through completion of the LOSS stage, 1 per week)

Breakfast:

1. One serving of lean meat, fish or eggs from the lists in the designated breakfast portion.

Lunch:

1. One serving of lean meat, fish or eggs from the lists in the designated lunch portion.

2. One serving of nuts, avocado or olives in the designated portion.

Dinner:

1. One serving of lean meat, fish or eggs from the lists in the designated dinner portion.

2. One serving of nuts, avocado or olives in the designated portion.

Seasoning, beverages, measuring food, and cooking per above.

### Calories

| Food Category | Breakfast | Lunch | Dinner | Snack | Sub-total |
|---|---|---|---|---|---|
| Protein | 130 | 185 | 185 | 0 | 500 |
| Nuts, Avocado | 0 | 190 | 190 | 0 | 380 |
| or Olives | | | | | |
| Oil & Seasoning | 0 | 10 | 10 | 0 | 20 |
| NTx | 25 | 35 | 40 | 0 | 100 |
| Sub-total | 155 | 420 | 425 | 0 | |
| Total | | | | | 1000 |

This period of a program embodying principles of the present invention can be very important for some participants, and can make the difference between a successful transition to a very low calorie diet with little or no hunger, and one with substantial hunger for as long as a week.

The Importance of Lipid Loading

The period of lipid loading ensures that the normal or immediate energy reserves available to the participant's body are adequate to sustain the body, and to help offset any hunger response that may occur across the first week. A sufficient reserve of normal energy will signal to the body the availability of calories, and helps preempt a response by the body to ingest more calories or store more fat. So, put simply, the greater the level of immediate energy reserves, the less the hunger in the first few days of the low calorie diet.

The phase of lipid loading lasts for a short time, e.g., two days. The participant will consume a higher level of fat than will be eaten throughout the program. These days will often have the side benefit of preparing the participant psychologically for a more restrictive diet. as with other phases or portions thereof, alcohol should be avoided. For those who find it hard to eat larger meals, it is often easier to complete the loading days by spreading food out into a larger number, e.g., six smaller meals across the day. It is also helpful to vary what the participant eats at each meal, as too much of anything can make a food unappealing.

Gaining Weight the Phase of Lipid Loading

Some participants will gain weight during the loading phase, and a fear of short term weight gain can be an impediment for some dieters to fully engage in the loading process. Therefore it is important to understand that for those who do gain weight during this phase, all of this excess weight should be lost very quickly, typically within the first few days of the very low calorie period. That said, many people will actually lose weight even during the of lipid loading phase for a variety of reasons. The Pace days are designed to keep the participant's weight loss on track by helping to avoid slow-downs or stalls in fat burning. The Pace days involve eating exclusively foods that contain dietary fats as a way of: maintaining intake of healthy fats that contribute to the weight loss and metabolic processes; diminishing carbohydrate intake levels to help ensure continued ketosis; and changing the eating patterns to which the body can become quickly accustomed. As such, incorporating Pace days into the diet regimen can help to prevent stalls in weight loss or plateaus that might be caused by the body's metabolic adaptation, slowed metabolism from fat intake restriction, or gradual reduction in the level of ketosis.

Pace days should be followed once per week throughout the Loss stage. During these days, the participant will: eat the same protein portions as in the Loss portion with the additional option of fatty fish (fatty fish portions are smaller than for white fish); eliminate all fruit, vegetable and greens portions allowed in the Loss phase; add the Pace food options of nuts, avocado and olives to the daily meal plan (two portions—one at lunch, another at dinner); and continue to follow the Loss guidelines for nutritional therapeutics, food preparation, measurement, cooking, beverages, sweeteners, sodium, seasonings and herbs. With the increased fat content during the Pace days, the calorie intake will be higher than typical Loss days—approximately 1,000 calories versus 820.

Across the course of the Loss phase of the program, the participant will be using a combination of pharmaceutical grade therapies to support the weight loss plan. Nutritional therapeutics are taken as indicated below:

AM dosage: NT4 (2 capsules); NT5 (1 capsule); NT6 (1 capsule); NT7 (1 capsule); NT8 (1 capsule); NT9 (1 capsule); NT10 (1 capsule); NT 11 (one scoop mixed with water daily (recommended Mid-AM); 1 scoop is equal to 1 level tablespoons or 3 level teaspoons).

Mid-day dosage: NT4 (1 capsule); NT5 (1 capsule); NT6 (2 capsules); NT7 (1 capsule); NT8 (1 capsule); NT12 (1 capsule); NT10 (1 capsule).

PM dosage: NT5 (1 capsule); NT13 (1 capsule); NT14 (1 capsule); NT7 (1 capsule); NT15 (2 capsules); NT16 (1 capsule); and NT10 (1 capsule).

| 10 Loss phase therapies | Regimen |
|---|---|
| Detoxification and Liver Support | NT9 |
| Insulin and Glucose Stabilization | NT5, NT4 |
| Endocrine Support | NT4 |
| Carbohydrate Craving and Mood Support | NT8, NT14 |
| Digestive Health and Colon Support | NT11, NT10, NT16 |
| Good Cholesterol and Anti-Inflammatory Support | NT15 |
| Hunger Support | NT11, NT12 |
| Fat Mobilization Support | NT7, NT13 |
| Energy Support | NT6, NT5 |
| Cortisol Stabilization | NT4 |

For many participants, intense exercise for the most part should be avoided during the Loss phase due to the significant caloric restriction. However, avoiding intense exercise does not mean leading a sedentary lifestyle. In fact, with a very small amount of consistent effort throughout the day, an “active” daily lifestyle will not only improve energy levels, muscle tone, and cardiovascular fitness, but will also improve your overall weight loss while practicing program embodying principles of the present invention. In the context of the present invention, one embodiment of “active” is walking 10,000 steps per day.

It can be particularly advantageous when a participant walks 10,000 steps per day, at least 5 out of the 7 days per week. Previous research performed at the University of Tennessee and Arizona State University found that the average person walks 5,000 to 6,000 steps per day for just normal activities. In addition, a moderate pace of constant walking yields anywhere from 3,000 to 3,600 steps when performed constantly for 30 minutes. Therefore, 10,000 steps is the equivalent to a normal day of activity plus a 30 minute vigorous walk. The health benefits of walking, both for cardiovascular health and for muscle and bone strength, can be achieved even when it is not done all at once. Therefore, as long as a participant varies activity throughout the day to equal 10,000 steps, the benefit is exactly the same as if done all over a much shorter period. Also, just for reference, 10,000 steps even taken casually equals between 300 and 400 calories burned per day. Another benefit to walking is that, if getting in 10,000 steps, it means the participant is spending less time sitting, as simply standing burns more calories than sitting, and sitting inactivates a person's natural fat burning enzyme lipase. By walking to this degree, a participant can ensure that they are spending as much time as possible with this enzyme breaking down fat.

Nutritional Therapeutics

NT11

A 14.83 gram dose of NT11 contains a blend of soluble and insoluble fiber totaling nearly 7.5 grams of fiber per dose, which is healthy for long-term maintenance of bowels, the blend of natural ingredients is specially formulated to be safe and effective, providing just the right amount of bulk for normal digestive system function.

Note: 1 scoop is equal to 1 level tablespoons or 3 level teaspoons.

14.83 grams contains

Proprietary Blend 13.73 g

Organic Psyllium Husk Powder*

Oryza™ Whole Grain Brown Rice Sweetener*

Psyllium Seed Powder*

Rice Bran*

Apple Pectin*

Fig*

Prune*
*% Daily Value not established

NT12

Fiber is an important part of a healthy diet, and NT12 helps supply the participant with extra soluble fiber. NT12 is a complement to a reduced calorie diet and daily exercise plan of the present invention. This all-natural supplement can help promote a feeling of fullness when taken with fiber-containing meals.

Serving size: 2 capsules

Calories 5

(Amount Per Serving, % Daily Value)

Total Carbohydrate 1 g<1%*

Dietary Fiber 1 g 4%*

Soluble Fiber 1 g**

Glucomannan 1,000 mg (1 g)***%

*Percent Daily Values are based on a 2,000 calorie diet.

**Daily Value not established

Other Ingredients: Gelatin, Rice Powder, Vegetable Magnesium Stearate.

As fiber products can affect the absorption of many medications, advantageously the participant does not NT12 within 2 hours of taking medications.

NT5

NT5 includes a multivitamin and mineral formula specializing in blood sugar and endocrine health concerns. It includes state-of-the-art, newly researched, anti-glycating agents such as carnosine, benfothiamine and pyridoxamine. The magnesium, chromium, zinc, manganese and vanadium are all preferably true chelates (available from Albion, Clearfield, Utah) with superior absorption.

Serving Size: 6 capsules (amount per serving, % daily value)

Vitamin A 3000 IU 60% (from fish liver oil and mixed carotenoids from palm tree fruit)

Vitamin C (as Ascorbic Acid) 500 mg 833%

Vitamin D3 (as Cholecalciferol) 400 IU 100%

Vitamin E 50 IU 90%

d-gamma tocopherol 100 mg

d-delta tocopherol 42 mg

d-alpha tocopherol 22 mg

d-beta tocopherol 3 mg

Vitamin B1 (as Thiamine HCl) 25 mg 1667%

Vitamin B2 (as Riboflavin) 25 mg 1471%

Vitamin B3 50 mg 250% (as Niacinamide)

Vitamin B6 50 mg 2500% (as Pyridoxine HCl and Pyridoxal-5-Phosphate)

Folates (NatureFolate™ blend) 400 mcg 100%

Vitamin B12 100 mcg 16667% (as Methylcobalamin)

Biotin (as d-Biotin) 4 mg 1333%

Pantothenic Acid 50 mg 500% (as d-Calcium Pantothenate)

Iodine (as Potassium Iodide) 75 mcg 50%

Magnesium 100 mg 25% (as Magnesium Chelazome®Bis-Glycinate Chelate)

Zinc 30 mg 200% (as Zinc Chelazome®Bis-Glycinate Chelate)

Selenium (as Selenium Glycinate Complex) 200 mcg 286%

Manganese 3 mg 150% (as Manganese Chelazome®Bis-Glycinate Chelate)

Chromium 500 mcg 333% (as Chromium Chelavite®Nicotinate-Glycinate Chelate)

Molybdenum (as Bis-Glycinate Chelate) 100 mcg 133%

Potassium 200 mg 6% (as Potassium Glycinate Complex)

Alpha Lipoic Acid 600 mg*

Taurine 600 mg*

Inositol 500 mg*

Green Tea (Camellia sinensis) (leaf) 200 mg*[standardized to contain 95% Polyphenols]

Carnosine 200 mg*

Benfothiamine 50 mg*

Vanadium 200 mcg*(as Vanadium Chelavite®Nicotinate-Glycinate Chelate)

*Daily Value not established.

NT4

NT4 includes a comprehensive endocrine balancing formula designed to promote optimal insulin, leptin and cortisol balance, and can be useful for anyone wishing to improve body composition. NT4 contains: the non-stimulating American ginseng for costisol balance; Banaba, chromium and vanadium to enhance glucose control and insulin performance; ForsLean™ (Coleus forskohli), Garcinia cambogia, and green tea extract promote lean body mass through ergogenic activity; Activated B vitamins, magnesium and L-carnitine provide nutrients for optimal mitochondrial function and metabolism; N-acetyl-tyrosine supports thyroid function and proper catecholamine balance; and GABA and magnesium, for helping to control cravings, and to help calm when stressed.

Serving Size: 4 capsules (amount per serving, % daily value)

Vitamin C (as Ascorbic Acid) 100 mg 167%

Vitamin B6 (as Pyridoxal-5-Phosphate) 5 mg 250%

Pantothenic Acid 100 mg 1000% (as d-Calcium Pantothenate)

Magnesium 10 mg 2.5% (TRAACS® Magnesium Glycinate Chelate Buffered)

Zinc 5 mg 33% (TRAACS® Zinc Glycinate Chelate)

Chromium 200 mg 167% (TRAACS® Chromium Nicotinate Glycinate Chelate)

Green Tea (Thea sinensis) (leaf) 300 mg*[standardized to contain 50% EGCG]

Foreslean® Coleus Forskohli Extract 250 mg*[standardized to contain 20% Forskohli]

Panax quinquefolius (American Ginseng) 200 mg*[standardized to contain 5% Ginsenosides]

Garcinia cambogia (fruit) 200 mg*[standardized to contain 50% HydroxyCitric Acid]

Banaba Leaf (Lagerstroemia speciosa) 100 mg*[standardized to contain 1% Corosolic Acids]

N-Acetyl L-Tyrosine 100 mg*GABA (Gamma-Aminobutyric Acid) 100 mg*

L-Carnitine (as Carnitine Fumarate) 100 mg*

Vanadium 100 mg*(TRAACS® Vanadium Nicotinate Glycinate Chelate)

*Daily Value not established.

Other Ingredients: Microcrystalline cellulose, magnesium stearate, silicon dioxide, rice flour.

NT13

NT13 helps support strong bones and teeth by increasing Calcium absorption and also assists in immune system health; helps maintain healthy bones in adults; and assists in maintaining a healthy immune system

Serving size: one capsule (Amount per serving % Daily Value)

Vitamin D3 5,000 IU 1,250% (as D3 Cholecalciferol)

Vitamin K 550 mcg 687% (as Vitamin K1 Phytonadione 500 mcg; Vitamin K2 Menaquinone-7 50mcg)

Other Ingredients: L-Leucine, microcrystalline cellulose.

NT15

NT15 supports healthy glucose levels, longevity, and the body's natural anti-inflammatory response. It includes double-strength EPA+DHA for increased omega-3 benefits in fewer soft gels, and provides exceptionally high levels of the omega-3 fats, EPA and DHA. It can be ideal for people wanting high intensity, therapeutic support in a smaller serving. NT15 supports healthy cardiovascular and respiratory function, joint flexibility and mobility, and the body's natural anti-inflammatory response.

Serving size: 2 softgels

Calories 18

Calorie from fat 18

(Amount per serving % Daily Value)

Total Fat 2.0 g 3%

Saturated Fat 0.1 g 1%

Trans Fat 0 g §

Vitamin E (dalpha tocopherol) 30 I.U. 100%

Omega-3s Weight Volume %

EPA (Eicosapentaenaic Acid) 650 mg 35%

DHA (Docosahexaenoic Acid) 450 mg 25%

Other Omega-3s 180 mg 10%

Total Omega-3s 1280 mg 70%

Oleic Acid (Omega-9) 56 mg 3%
*Percent Daily Values are based on a 2,000 calorie diet.

§ Daily Value not established

Ingredients: purified deep sea fish oil (from anchovies and sardines), soft gel capsule (gelatin, water, glycerin, natural lemon oil), natural lemon oil, d-alpha tocopherol, rosemary extract.

NT6

NT6 provides the participant with a natural energy source, and contains essential fatty acids and Medium Chain Triglycerides (MCTs). MCTs are oxidized differently in the body than other fats, making NT6 a readily available energy source.

Serving size=2 soft gels

(Amount per serving % Daily Value)

Calories 25

Calorie from fat 25

Total Fat 2.5 g 4%*

Saturated Fat 2.5 g 12%*

Proteins <1 g*

Organic Coconut Oil 2,000 mg (2 g)*(Cocos nucifera) (fruit) which typically contains:

Lauric Acid 880 mg**

Myristic Acid 260 mg**

Caprylic Acid 106 mg**

Palmitic Acid 150 mg**

Capric Acid 90 mg**

Oleic Acid 100 mg**

Stearic Acid 20 mg**

Linoleic Acid 20 mg**

*Percent Daily Values are based on a 2,000 calorie diet.

**Daily Value not established.

Other Ingredients: Gelatin, Medium Chain Triglycerides, Glycerin, Titanium Dioxide Color.

NT7

NT7 supports a healthy body composition, including exercise and dieting support. Studies show that CLA (Conjugated Linoleic Acid) can help support dieting goals and a healthy body composition.

Serving size: 2 softgels

(Amount Per Serving % Value Daily)

Calories 30

Calories from Fat 25

Total Fat 3 g 5%**

Protein <1 g 1%**

Myoleptin™ CLA 3,000 mg (3 g)***(from Safflower Oil)

Contains 78-84 percent 2,340 mg-2,520 mg Conjugated Linoleic Acid (CLA)

**Percent Daily Values are based on a 2,000 calorie diet.

***Daily Value not established.

Other Ingredients: Gelatin, Vegetable Glycerin, Natural Caramel Color.

NT9

Serving size: one softgel (Amount Per Serving % Daily Value)

Milk Thistle (Silybum marianum) 250 mg*(seed) (from a 4:1 Extract)

(Equivalent to 1,000 milligrams of Milk Thistle)

*Daily Value not established.

Other Ingredients: Soybean Oil, Gelatin, Glycerin, Soy Lecithin, Vegetable Shortening, Yellow Beeswax, Caramel Color, Titanium Dioxide Color.

NT8

NT8 can increase neurotransmitters, which can be helpful for food cravings, appetite control, smoking cessation, insomnia, depression, anxiety, mood swings, premenstrual syndrome, obsessive compulsive disorder, addictions, stress, and Type II Diabetes. NT8 can naturally support neurotransmitter production. Many factors have an impact on brain neurochemistry and endocrine balance, such as age, genetics, circadian rhythms and various environmental factors including stress, diet, exercise and medications. Many people do not synthesize adequate serotonin including diabetics and those on SSRI's for a long period of time. Dieting is known to lower levels of tryptophan, an amino acid needed to make serotonin. Dieters, and others, often need help in controlling food cravings, mood swings and addictions. Cravings for cigarettes are known to be controlled by Dopamine receptors. Dopamine is another very important neurotransmitter often low in those who are prone to addictive or erratic behavior. Imagine what might be thrown off if one or more of these important neurotransmitters is deficient.

The perception of hunger is determined by many physiologic factors, including:

1. Neurotransmitter balance of Serotonin, Dopamine, GABA, Glutamine, Acetylcholine and opioids. In general, any deficiency of these could trigger anxiety, hunger, depression, and cravings.

2. Genetic specificity of brain neurochemistry and hormonal balance.

3. Body fat stores. Adipose tissue produces various metabolic signals that affect the appetite: leptin helps to control hunger, inflammatory cytokines (TNF-alpha, IL-6, etc.) increase it.

4. The level of glycogen stores in the liver is communicated to the brain through the vagal nervous system, thus attempting to influence the appetite.

5. Blood glucose levels are perceived directly by the brain through glucoreceptors. Low blood glucose levels trigger carbohydrate cravings.

6. Adrenal hormones, cortisol and adrenaline, increase hunger and the production of sex hormones (estrogen, testosterone, progesterone). These all influence appetite.

7. Gut derived hormones are produced directly in response to the macronutrient composition and size of the meals: Cholecystekinin (CCK), in response to protein, fat and the stretching of the stomach by food/drink volume; Galanin, in response to fat; and Ghrelin, in response to an empty stomach. They signal the brain directly. CCK and galanin reduce hunger while Ghrelin stimulates it.

8. Pancreatic hormones: high insulin levels signal the brain to reduce hunger if the tissues are insulin sensitive.

The amino acid L-Tryptophan (LT) converts into 5-Hydroxytryptophan (5-HTP) which converts into 5-Hydroxytryptamine (5-HT), also known as serotonin. 5-HTP readily enters the blood brain barrier and makes serotonin. Oral administration of 5-HTP has been shown to successfully raise serotonin levels. Serotonin is known to control sleep, depression, anxiety, aggression, appetite, temperature, sexual behavior and pain sensation. Other neurotransmitters and CNS chemicals, such as melatonin, dopamine, norepinephrine, and beta-endorphin have all been shown to increase following oral administration of 5-HTP. This ability to increase not only serotonin levels in the brain, but also dopamine and norepinephrine, allows 5-HTP to produce some significant and unique effects on brain chemistry and on serotonin-related conditions which other substances, including L-Tryptophan, cannot duplicate. L-Tryptophan may raise serotonin levels or may be shunted into the synthesis of niacin or the production of protein.

Research shows that Type II Diabetics have lower brain tryptophan levels and higher rates of depression. This may help explain their propensity towards sugar addiction beyond the typical fluctuating blood glucose levels. When twenty overweight, NIDDM participants were given 750 mg/day of 5-HTP or placebo for two weeks in a double-blind study, their daily energy intake decreased considerably as well as their carbohydrate and fat intake. Body weight also diminished along with appetite.

NT8 includes, per serving (mechanisms of action of brain nutrients)

Vitamin C=100 mg (Cofactor in adrenaline synthesis)

Vitamin B6=20 mg (Cofactor in serotonin and dopamine synthesis)

Vitamin B12=25 mcg (Cofactor in serotonin and dopamine synthesis)

Niacinamide=20 mg (Cofactor in brain energy production)

L-Tyrosine=1,000 mg (Precursor to dopamine and adrenaline, Antidepressant, Reduces cravings for tobacco)

5-Hydroxytryptophan=100 mg (Precursor to serotonin and melatonin, Reduces carbohydrate cravings and appetite, Corrects age-related decline in serotonin)

Taurine=50 mg (Important regulator of calcium and neurotransmitters within the heart, muscles and brain, helps calm the nervous system by regulating neurotransmitters, strengthens and protects healthy cell membranes)

Rhodiola Rosea=50 mg (Improves levels and metabolism of beta endorphin, dopamine, and serotonin; Adaptogen action, helping with stress)

NT10

(Amount Per Serving % Daily Value)

blend of enzymes 156 mg**

Amylase 3,500 DU**

Protease 4.5 15,000 HUT**

Invertase 150 SU**

Protease (6.0 conc.) 7,500 HUT**

Glucoamylase 6 AGU**

Protease 3.0 13 SAPU**

Alpha-galactosidase 110 Gal U**

CereCalase™ 200 MU**

Malt Diastase 200 DP**

Lipase 125 FCC LU**

Cellulase 250 CU**

**Daily Value not established

Other Ingredients: rice bran, vegetable cellulose, water.

NT16

Serving Size: 2 capsules

(Amount per Serving % Daily Value)

Tribulus terrestris 400 mg**[standardized to contain 40% furostanol saponins]

Sweet Wormwood (Artemisia annua) (stem and leaves) 300 mg**[standardized to contain 10% artemisinin]

Magnesium Caprylate 300 mg**(yielding 267 mg Caprylic Acid; 23 mg Magnesium)

Berberine Sulfate (from Berberis aquifolium) 200 mg**

Grapefruit Seed Extract (Citrus paradish) (seed) 200 mg**

Barberry (Berberis vulgaris) (bark) 100 mg**[standardized to contain 6% berberine]

Bearberry (Arctostaphylos uva ursi) (leaf) 100 mg**

Black Walnut (Julglans nigra) (hull/leaves/bark) 100 mg**

**Daily Value not established

Other Ingredients: Microcrystalline cellulose, magnesium stearate, rice flour.

NT14

NT14 includes (serving size=2 capsules)

(Amount per Serving % Daily Value)

Vitamin B6 10 mg**(as Pyridoxal-5-Phosphate)

Valerian Root (Valeriana officinalis) (root) 400 mg**[standardized to contain 0.8% valerenic acid]

Passion Flower (Passiflora incarnata) (aerial) 200 mg**[standardized to contain 3.5% flavonoids]

Lemon Balm (Melissa officinalis) (leaves) 200 mg**[standardized to contain 5% rosemarinic acid]

German Chamomile (Matricaria recutita) (flower) 200 mg**[standardized to contain 1.2% apigenin]

Gamma Amino Butyric Acid (as PharmaGABA®) 100 mg**

L-Theanine 100 mg**

5-HTP (5-Hydroxytryptophan) 100 mg**

Melatonin 3 mg**

**Daily Value not established

Other Ingredients: Microcrystalline cellulose, vegetable stearate, silicon dioxide.

Benefits of Priming Before a Meal

There is a growing body of evidence that the practice of taking in a relatively small amount of very low calorie (or calorie-free) drink or food prior to your actual meal can provide benefits to your heart and your weight; this practice is called priming. Multiple studies through the years have shown a clear benefit to having something filling prior to each meal. By filling the stomach before an actual meal is eaten, the stretch receptors in the stomach begin to get stimulated early. It is commonly said that it takes about 20 minutes for your brain to get the message that you're truly full. At the furious pace that many of us eat (especially at work, or in front of the TV or computer when we're really not paying attention), 20 minutes can equate to a huge amount of extra food that we're able to take in prior to the signaling to be acknowledged by the brain. This can lead to an unnecessary caloric excess at every meal, and can account for long term or continuous weight gain.

When we start stretching the stomach early, however, we give time for the corresponding chemical and electrical signals to get to the brain in sufficient quantity to “prime” the satiety response. This allows one to become full with the actual meal that is subsequently ingested in a much earlier time-frame, allowing a decrease in the total amount of food taken in, a decrease in calories, and greater ease in losing and maintaining weight without feeling hungry or deprived. Various studies have looked at multiple foods for reducing hunger prior to meals—from plain salads to soups, water, fiber, and fruits such as apples and grapefruit. In reviewing this research, new evidence from the March 2011 Journal of Nutrition and Metabolism found that while there was no real difference in the amount of calories consumed or the amount of weight lost when using grapefruit, grapefruit juice, or water prior to meals, there WAS a difference in HDL cholesterol for those individuals consuming the grapefruit or grapefruit juice. In fact, this resulted in a roughly 6 to 8% improvement in HDL (generally considered healthy cholesterol) levels.

It's too early to know exactly why this benefit occurred. It could be because of the fiber inherent in the fruit, the vitamin C, both of those factors, or something else. However, it can be advantageous know that something that helps control appetite can also have a direct effect on heart health. Because of the repeated benefits researchers have documented from the practice of pre-meal hunger management, and because of its simplicity, another optional aspect of a program embodying principles of the present invention includes the use the priming before a meal to help control hunger and decrease total energy intake for improved weight loss. Recognizing that hunger may not be a major issue for any particular participant during a program as described herein, due to the NTx regimen and the nutrient structure and fiber of the prescribed meals, adopting the habit of priming during a program and beyond could afford long term benefits even after reaching a weight loss goal, helping to prevent weight regain. Thus, priming during a program as described herein builds yet another healthy habit that can improve chances of long term weight management success.

Minutes Prior to Each Meal

Consume one of the following items approximately 20 minutes prior to each meal*—

4 ounces of unsweetened grapefruit juice mixed with 4 ounces of water (8 ounces total)—mixing the grapefruit juice with water reduces the glycemic impact and increases satiety

½ grapefruit—remove the outer rind of course, but don't “scoop” the fruit out. Get all of the benefits of the fiber from the skin that surrounds each wedge

8 to 10 ounces of water—ideally ice water—your body having to warm the water burns more calories

Grapefruit and/or grapefruit juice preferably is selected for no more than two of three meals daily, and each will count as the fruit portion that is allotted in the dietary guidelines. As such, priming for one of three meals each day advantageously will be with water only; water may be chosen for up to three meals daily.

At Meal Time

Eat the portion-controlled meal as normal. Remember that eating the grapefruit or drinking grapefruit juice will use up one of your daily fruit allotments each time. Priming before a meal does not allow the participant to have extra fruit portions beyond the dietary guidelines—it only impacts the timing of those portions (before instead of with or after the meal).

While most participants are able to comfortably sustain a low calorie diet across a program as described herein, there may be brief points in time or even several day periods, when hunger seems more acute. Below are a dozen strategies to help manage hunger so that the participant can successfully maintain the dietary program.

1. Spread Food Out Across the Day:

The participant will quickly note your hunger pattern across the day—perhaps one is hungrier in the morning than the afternoon. It is okay to shift, for example, a piece of fruit from lunch to the morning. Similarly, the three meals can be broken up into six separate occasions (without increasing total food intake).

2. Drink Water

Often times the human body will confuse thirst for hunger, and as such it makes sense to always drink a glass of water as the first response to hunger before eating. Wait 10-15 minutes, and then reassess.

3. Paced Eating:

It can take time for the human body to fully register that it has ingested food, which means that one can eat more than they need to satisfy hunger if they eat quickly. Having some portion of your allocated food—while chewing slowly and appreciating every bite—then waiting 20 minutes to reassess hunger is a good discipline to make the most of food while minimizing hunger.

4. Keep Up the Fiber Intake:

In addition to helping with digestion, consuming fiber contributes to a feeling of fullness. Leafy greens included in the program are a great source of dietary fiber at meals. The fiber supplements (in conjunction with at least 8 to 10 oz of water to make the fiber expand in your stomach) may be timed across the day so to help maintain the sense of fullness.

5. Be Aware of Visual and Olfactory Cues:

Humans have a normal hunger response to both the sight and smell of food, which is, for example, why the smell of cooking food can make one hungry before mealtime. This means that exposure to food between meals can contribute to an unusual feeling of hunger, at times when one might otherwise not have any hunger at all. Minimizing this type of exposure, or at least understanding that this response is temporary, is therefore helpful in also minimizing hunger. Related, using a small plate rather than a large one can make you perceive portions as larger, and therefore more satisfying.

6. Be Aware of Social Eating:

Studies show that people will consistently eat more in the presence of others than they do alone. While it may be impractical to avoid eating with others, maintain self-awareness about the amount and type of food you are eating, and minimize the duration of social meals.

7. Be Aware of Habitual Eating:

We are all creatures of habit, especially when it comes to the types and timing of food that we consume, and many occasions for eating relate more to the pace of the day than to true hunger. Perhaps one eats regularly before bedtime because they are relaxed and see food as a reward, or maybe one eats in the mid-afternoon or at other points in the day because they are taking a break from work, or every time one sits in front of the television. A program as described herein is an opportunity to identify these times of habitual eating, and to discontinue this behavior.

8. Avoid End-of-Day-Eating With Some Extra Sleep:

For late night eaters (after 7:30 pm), food consumption may in fact be contributing to staying awake longer, as the body gets re-energized. To break this habit, a reliable tactic is to simply go to sleep earlier—it is extremely unlikely that you will wake up due to hunger.

9. Be Aware of Emotional Eating:

There are many emotional triggers for eating, such as stress or anxiety. It is important to realize that these emotional triggers will not simply disappear by virtue of embarking on a diet, so someone can actually perceive hunger for psychological reasons even when they do not physically need additional calories. Self-awareness is the key to identifying this emotionally driven hunger, and to consciously substituting other behaviors or activities for emotional eating. These activities might include social interaction, writing in a journal or reading—things that increase a sense of well being and connection, while also helping to maintain a balanced emotional state. Jotting down why one is eating can help the participant become more mindful.

10. Stay Busy

Everyone has experienced times when a full schedule has caused them to work through meals without noticing. By focusing time across the day on productive activities—and avoiding any obsessing over food—it is much easier to maintain a diet. Scheduling and planning the day, which are good habits in and of themselves, can be helpful in making sure that the participant is not idle or in situations where it is too easy or convenient to focus on eating.

11. Rate Hunger

Get into the habit of evaluating hunger level across the day, and especially before and after each meal. When the participant sits down to eat a meal, the participant should be hungry, but not ravenous. Letting the blood sugar get too low often leads to binge eating, and overconsumption of calories. Here's a numeric scale to use—

9-10 Ravenously hungry, salivating.

7-8 Hungry, belly growling.

5-6 Mildly hungry; may need a light snack, but could hold out a little longer.

3-4 Satisfied, don't need to eat anymore.

1-2 More than satisfied; ate too much.

0 Stuffed

12. Visualize and Document Goals and Accomplishments:

A program as described herein will facilitate rapid weight loss, which creates a virtuous cycle of behavior change leading to increasing, positive results. As part of the plan, a participant should see advancements each day on the scale and in the mirror. Write down this progress (weight, measurements, clothes size), and in moments of temptation, refocus on a daily and overall weight loss goals—specifically recall the discipline and eating approach that allowed the participant to achieve. Though the hunger might be acute at particular points during the program for a variety of reasons, in general hunger should be manageable across the diet. From an expectations standpoint, having little or no hunger whatsoever may be possible for some, but most will have a low to moderate level of hunger that is sustainable if managed correctly.

Here are some questions to ask the participant to help identify compliance issues or strategies that might improve hunger management.

1. Have you eaten all food components in the correct quantity, and have you done a calorie count? It is possible that you could be eating less than prescribed number of calories by virtue of serving size or type of foods selected, which would definitely add to your hunger. As such, it is best to do a calorie count retrospectively for the foods in your food diary and on an ongoing basis to verify calorie intake. Also, if you are leaving out components such as vegetables or leaf greens that are a source of fiber, this (along with not drinking enough water after your meal) could reduce the feeling of fullness after your meals—as well as cause a nutritional deficit that stimulates hunger.

2. Have you tried spreading out your food across the day? If helpful in managing your hunger, you can have your fruit between meals.

3. What are your ketone readings and what is your loss to date? If you are not in ketosis and you are not losing weight, this would suggest that you may be eating foods that are high in fat or carbohydrates, beyond the program guidelines. This should be cause for a thorough review of compliance with all program elements, but especially food, beverages and anything used in cooking or for sweetening.

4. Have you eaten any foods/beverages/seasonings that are not expressly allowed, especially anything with carbohydrates or sugars? Carbohydrates will trigger a hunger response soon after eating, as insulin will be released and quickly reduce the level of sugar in the bloodstream—which in turn creates hunger. Unfortunately, sugar is hidden in many foods (including seasonings), so you could be inadvertently sabotaging yourself—read all labels carefully for ingredients (don't just read the headlines about fat-free, etc.).

5. Are you eating organic and preparing your own food? Many non-organic meats including beef and chicken breasts are injected with a sugar solution by the company before packaging to give a more palatable (and some say addicting) taste. This is another reason we suggest strongly eating organic free range meats, as they have not been “doctored” in this way. Similarly, most restaurants will add a glaze or other flavorings that commonly have sugar, which is why we suggest preparing your own food whenever possible.

6. What is your activity level, and are you walking 10,000 steps—or more? If you are extremely active or exercising beyond the target level of walking, you might be creating a larger calorie deficit for your body than might normally be expected during this program, which in turns generates greater hunger (and potential resistance to fat loss that will slow your metabolism). This may necessitate pulling back a bit on activity across the remainder of the program. This has to be balanced with another trigger for hunger—inactivity or boredom. If you find that you are hungry when just sitting around, get up and move for 5 to 15 minutes. For many people, this will help make that “boredom” hunger disappear.

7. Are you drinking the prescribed amount of water? Water adds to the feeling of fullness (especially when combined with fiber), and facilitates the transport of fat and nutrients from that fat. As such, drinking too little can impair weight loss, while also exacerbating hunger. Do your best to drink water continuously across the day.

8. Is your hunger generalized, or is it focused on particular foods? If particular foods, what are they? Most of the suggestions above relate to generalized hunger. However, sometimes cravings, especially for carbohydrates, point to issues of low serotonin levels (carbohydrates can increase these, and are often craved when serotonin is low), bacterial imbalance in the stomach and intestines, and/or the presence of Candida or other fungus in the colon (these bacteria and fungi burn sugars, and in the absence of these sugars will release toxins in the body to create intense cravings).

9. Do you have hunger at particular times of the day, or in certain situations? There are often habitual eating times—for example, mid-afternoon or late evening—when people eat for reasons other than hunger (boredom, as a break in one's schedule, to relieve stress, etc.). Such habits can carry over into the program, and it may take a week or more to become aware of and break these habits such that hunger dissipates at these times. Other common situations include eating around colleagues or family members in social situations, or simply getting hungry when in the presence of food. It can be best to avoid circumstances such as this if you know that this triggers acute hunger—these are basic stimulus-response situations that are often outside of our conscious control.

10. What are your hunger ratings across the day (1-10)? (see above for scale definitions) In order to understand the relative strength of your hunger across the day, and from day to day, it is important to track hunger. Patterns that you observe can help you in applying the hunger management strategies, and in identifying changes in hunger that may be linked to different aspects of the program or your daily activities. If you note a consistent “10” at particular times of the day, you may want to save fruit to eat at that time.

11. Are you drinking fluids as your first response to hunger? A glass of water or cup of tea can often reduce hunger between meals or in the morning before your first meal. Successful dieters typically drink fluids continuously across the day. Combining one of your doses of fiber with the fluids will have a synergistic effect on filling the stomach and satiating hunger.

12. Are you pacing your meals? It takes at least several minutes for your body to register satisfaction from recent food intake, so best to eat slowly and to even pause briefly across your meal to allow the satiety response to occur.

13. How much sleep are you getting? Those who sleep 6 hours or less per night have higher hunger levels (16% higher levels of the hunger stimulating hormone Ghrelin) and require more food to be satisfied than those who sleep more (15% lower levels of the hunger reducing hormone Leptin). If you are not sleeping 6 hours or more (ideally 7-8 hours) each night, you could likely reduce your hunger with more sleep.

14. How would you characterize your stress levels? The hormone Cortisol, which is produced by your body in response to stress, is a pro-storage substance that contributes to the accumulation of fat and in turn weight loss resistance. Reducing or better managing your stress can be helpful in maximizing the weight loss process during this program and beyond.

15. How soon after eating do you get hungry again? If you are hungry immediately or very soon after eating, you should first examine your food portions to be sure you are eating adequate calories and fiber, including all diet components. If the diet is correct, this can be a red flag for gut health issues related to bacterial balance or yeast overgrowth (and cravings especially for carbohydrates, even if you have just eaten). A program as described herein can provide supplementation that aids in resolving this issue.

16. Are you depressed or bored? For many individuals, these feelings are strong motivators for food intake. If this has been an issue for you in the past, it can be a very difficult pattern to break, as this particular mind-body connection has often been reinforced for year. A first step to help in these situations is to get outside and walk for at least 10 minutes as your initial response to each and every substantial hunger urge that you experience outside of your three scheduled meals. By doing this, you are “resetting” the body's response to boredom or depression by making your mind crave movement to deal with these emotions rather than food. Also in support of this approach, there is significant research across multiple studies to show that one of the most effective short and long-term strategies to combat depression is exercise (even simple walking is enough).

Understanding and Controlling Carbohydrate Cravings

A craving can be distinguished from general hunger by the specificity of the desire for a particular kind of food, and by the times when cravings occur—which are not necessarily in sync with the times when the body otherwise needs caloric intake. While generalized hunger especially at or around meal times is a normal signal by the body to seek a broad range of nutritional requirements, carbohydrate cravings can be a signal of much more specific nutritional or biochemical need. One of the primary reasons for carbohydrate cravings is a low level of serotonin, the hormone that brings a feeling of wellness. Studies have demonstrated that consumption of carbohydrates can cause a short term spike in serotonin, and as such, your body will crave carbohydrates as compensation for low serotonin. These cravings occur even though your body does not necessarily need the associated calories or other nutritional components of the high carbohydrates foods that you may eat on those occasions. For this reason, cravings can feel similar to “addictions”, where the body develops an acute desire that is hard to resist, even for those with above average willpower.

Controlling Carbohydrate Cravings

In general, dieters who follow a program as described herein can see the elimination of cravings during the diet period. Over the long term, the best way to minimize cravings is to, consume a diet with low glycemic index foods, eat in small portions across the day, which helps to keep a consistent level of blood sugar, and exercise and build muscle, which aids in the storage of insulin to regulate blood sugar levels in response to food intake.

Supporting Serotonin Production

During a program as described herein, a participant you may experience carbohydrate cravings as they become accustomed to the low glycemic diet that have been outlined. To help in managing and resisting cravings until the participant has become adjusted to the low glycemic regimen, another optional aspect of a program embodying principles of the present invention includes a product that facilitates the production of serotonin without the need for excess carbohydrates.

The Loss Stage: a Ketogenic Diet

A “ketogenic diet” refers to an eating regimen that causes the body to burn fat as energy—or a state of “ketosis”. The fuel most commonly used by the body on today's typical American diet is carbohydrates, and it is only in the absence of carbohydrates from the daily diet or from stores of glycogen (readily available energy in the body) that one will enter ketosis. The by-product of fat burning is “ketones”, a form of energy that is also readily used by the body. The ketones produced through ketosis are not dangerous or toxic in the long or short term, though some mistakenly believe this to be the case due to confusion with diabetic ketoacidosis, a condition with many aberrant metabolic processes that does not occur on a ketogenic diet. Ketogenic diets are now in fact used in a number of therapeutic applications, particularly in the case of neurological illnesses, with such diets having been shown to allow substantial or even complete improvement in certain symptom patterns where all other medications and even surgical procedures have failed.

Achieving and Maintaining Ketosis

As noted, ketosis can only occur in the absence of carbohydrates in any meaningful quantity. Most people will enter and maintain ketosis by consuming a daily diet with carbohydrate intake of 40-60 grams, though some can achieve ketosis with as much as 80-100 grams and still some others more rarely require restriction to as little as 10-20 grams. This is one of the reasons that grains, processed sugars and high carbohydrate foods, are advantageously excluded from the Loss stage of the present program—and that the allowed carbohydrates will generally come from food in its natural, more fiber dense form which slows the process of carbohydrate assimilation while making the body work harder to extract this energy. It is also why seemingly small “cheats” with carbohydrate rich foods can kick the body out of ketosis. Temporary periods without ketosis due to such one-time deviations from diet guidelines can last for up to several days, while continuous consumption of carbohydrates beyond program guidelines can potentially result in much lower or almost negligible fat burning during the program period.

### Measuring Ketosis

The level of fat burning or ketosis will generally correspond to the level of ketones circulating in—and eliminated by—the body. As such, as a proxy for measuring ketosis, it is useful for participants to measure the level of ketones present in their urine across the program. Ketone measurement strips, commonly commercially available, provide the participant with an indication on a daily basis that they are in ketosis. It is recommended to take daily readings first thing in the morning starting with the low calorie portion of the program (after loading).

Negative and Low Readings with Weight Loss

There are several factors that can cause readings to appear negative or light even when the body is indeed losing weight and burning fat, including:

Excessive water consumption that dilutes ketones by causing a larger volume of urine. If above the suggested intake of 2 liters per day, the participant may be causing the ketone concentration to be lower, masking the true level of fat burning. However, if the participant is thirsty, they should drink and not worry about ketone measurement if they are still losing weight.

A high level of efficiency in burning ketones before they are eliminated from the body, creating the perception that ketosis is not occurring (this varies by individual and is not predictable in advance, but again is not of great concern if weight loss persists). In other words, the body is producing plenty of ketones for energy, but it is using such a high percentage for energy that there is very little left over to spill into the urine as waste.

In such circumstances, the presence of weight loss is the most important indicator of progress, and as long as such loss continues, one need not be alarmed by negative or light ketone readings.

Negative and Low Readings without Weight Loss

When coincident with minimal or no weight loss for an extended period, light or negative readings are a sign that carbohydrate and/or total calorie intake is too high. Such readings are most likely due to (inadvertent or purposeful) consumption of calories/carbohydrates in excess of program guidelines. The exception to this is during a woman's monthly menstrual cycle, which can cause readings to be lighter or negative (positive readings typically resume shortly after the cycle is completed).

Ultimately, it is virtually impossible to follow the dietary guidelines as described herein and not be in ketosis within two weeks of starting the low calorie phase. One cannot be on a very low carbohydrate diet and also not shift into ketosis. The human brain can only use 2 types of fuel to survive—glucose, and ketones. An absence of both of these substances is not compatible with survival. If a participant is not in ketosis after 2 weeks, and is not losing weight, then there is some aspect of food intake that is not correct, as the participant is undoubtedly burning glucose from some food source.

Timeframe for Entering Ketosis

While most will enter ketosis within 3-5 days of starting the low calorie phase of a program as described herein, it is possible that ketosis may not be evident for 10-12 day and occasionally even longer. The pace at which one enters ketosis involves several variables including: the level of stored glycogen in the body, which is drawn down by the body before fat burning begins; the level of activity and caloric deficit—the greater the deficit, the faster one will enter ketosis; the percentage of body fat (studies show that those with a higher percentage of body fat will take longer to enter ketosis, often more than 10 days), which this could be due to high glycogen levels, metabolic resistance to fat burning, or other factors; and the efficiency of one's body in using ketones for energy, and therefore the level of excess ketones that are excreted through urination.

If a participant finds that weight loss has stalled during the program, this can be expected. Plateaus are expected—weight loss can vary from day-to-day or even from week-to-week for a host of reasons, and often one can be losing fat while not losing weight due to the effect of water replacing that fat volume for a short period of time until the body readjusts. However, if the program is being followed perfectly, the ketone sticks have not changed meaningfully from when the participant was consistently losing weight, the participant is drinking at least two liters of water daily, the participant is getting at least six hours of sleep each night, and there is no noticeable weight loss in five days or more, below are some strategies that may be able to get the scale moving downward again. It is recommended that these strategies be tried individually and in the order listed below.

More Water

Some people simply aren't losing weight because they don't consume enough water. Not only is water necessary in the enzymatic reactions to allow the liver to break down fat and use it for energy, but the body, not receiving many calories, will begin to hoard water if it does not sense a significant influx to maintain its needs, preventing the participant from seeing a loss at the scale. If one is not drinking at least two liters of water daily, the first step is to increase water consumption. This alone may be enough to restart your weight loss.

Increase Fiber

Sometimes weight gain can be not only from retaining total body water, but also retaining water and other material in the colon. For some individuals, simply doubling fiber intake can help to move this excess out of the colon and allow for continued weight loss. If one has had difficulty tolerating the fiber at the regular dose (some people tolerate this form of fiber better than others), this may not be an acceptable option. If one finds this to be effective, they should return to the normal dose of fiber once they have started losing again. If they find that they hit another plateau, they may try added fiber again at any time to see if it will have the same positive effect

Light Exercise

If the participant haven't been walking at least 10,000 steps every day, now is a good time to start doing that. This simple addition alone could be all they need to start burning fat at a higher level. They don't have to do it all at once. In fact, one of the key components of simply walking 10,000 steps is that it takes a while, and is best accomplished by staying active and moving throughout the entire day—if walking, then that means the participant is not sitting. Sitting is the enemy to fat loss, as merely resting in a seated position inactivates Lipase, a key enzyme necessary for fat burning. Simply getting up and moving removes a significant barrier to fat loss.

If one has been walking at least 10,000 steps in every day, then trying some exercise at a higher level may make a difference. One needs to monitor how they feel, and make sure they have sugar (like an apple) with them should they feel light headed. Adding light weight lifting, running, or some other type of more vigorous exercise (within the limitations of one's health set forth by a physician) may kick-start the fat burning engine and allow continued weight loss. Once one begins to see weight loss again, it is advantageous to return to 10,000 steps per day. If the participant finds that they plateau relatively quickly, they may be one of the few people that require some form of more vigorous, more regular exercise. If this is the case, they may continue with this level of activity as long as tolerated without issue.

Apple Day

Instead of starting the day with tea or coffee in the morning as normal, a participant could only have water and the fiber supplement. After this, the participant will begin eating apples, and only apples, for the remainder of the day. Whenever hungry, the participant is allowed to eat a large apple. They can do this only when they are hungry, or they may space them evenly throughout the day (especially effective for people who find they have energy dips or get a bit “cranky” if they don't have a regular supply of sugar in their system). They can have a maximum of six apples for the entire day, though they are allowed to eat less if they are not hungry. During an apple day, one can only drink water; no other beverages are permitted, and they should only drink enough to quench your thirst—no more. While this is counter to the general recommendations regarding regular water intake, but for many people the issue with stagnant weight loss is merely continued water retention, and this apple day will help the participant's system to purge some of that excess water. The apple days should be done for one day only. It is not meant to be done on successive or multiple days throughout the program.

Steak Day

The participant has tea or coffee for breakfast as normal, and then have only water, tea and/or coffee for lunch. Do not eat any food until dinner time. At dinner, eat only a large, lean steak (6-9 ounces or 170-255 grams, pre-cooked weight) selecting from the approved cuts of beef, or pair the steak with a small apple. Do not eat anything else. Ensure that the participant drinks at least two liters of water for the day. Having a single meal that is almost purely of protein is another way to stimulate the body to flush extra water that it may be retaining, which could be artificially inflating weight. Do not use this strategy any more than 1 day out of any 2 week period.

Key Factors in Weight Loss Resistance

The primary cause of obesity in the United States is overeating combined with a lack of physical activity. Unfortunately, once people store excess body fat, they often have significant difficulty shedding those added pounds, even if they become truly disciplined in both their dietary and exercise habits. Below are some of the major reasons for this difficulty.

Metabolic Syndrome

The body was designed to allow us to cope with significant short term stress by releasing a flood of hormones that help us to escape danger. All the things that we need to run away from an attacking predator—an increased heart rate, increased blood pressure, increased blood flow to muscles, decreased blood flow to the intestines and kidneys and decreased sensitivity to insulin—are part of the stress response, and wonderful for short bursts of five to ten minutes. Unfortunately, our lives and environment have evolved much faster than our bodies, and the very same response that was designed to protect us in the short term can instead become incredibly damaging in the long term. When we are upset by a situation at work, at home, on the highway, or in the grocery store, these situations will commonly elicit the very same short term stress response that occurs when we are in real danger—even though these are clearly not life or death circumstances. As such, the aggravations of our modern daily environment can transform a response that was meant to occur only occasionally into an almost continuous state. What should be temporary physiological stress responses (like increased blood pressure and decreased insulin sensitivity) now set in as permanent changes—a condition commonly referred to as Metabolic Syndrome.

With the body's decreased sensitivity to insulin from repeated stress response, the pancreas needs to release more and more insulin over time to compensate for the continuous stress level. Since insulin is a “pro-storage” hormone—meaning that it tells your body to store fat for times of famine—this elevated level of insulin also signals the body to store more and more fat. Compounding the problem, the body will now also produce an increasing level of estrogen from the greater amount of stored fat (which contributes to even further fat storage), while producing a decreasing level of the hormone DHEA (which in turn decreases testosterone, a hormone that contributes to fat burning). Finally, inflammation begins to increase throughout the entire body, leading to vascular changes, ulcers, decreased bone density, decreased immunity and an increased risk of various cancers. In this way, a simple stress response cascades into Metabolic Syndrome, a multi-pronged hormonal battle against weight loss and your health overall.

Environmental Stress and Toxins

Our environment is full of toxins that stress the body and its own internal detoxification systems. Metals such as mercury, aluminum, and lead, pesticides like DDT, DEET, and organophosphate, herbicides, ethanol, smoking, PCB's, dioxins, phthalates, trans-fats, and many drugs are ALL toxic to our body in general, or toxic to specific processes in our body necessary for proper functioning. Unfortunately, many of the non-organic foods that we eat can contain these toxins. While our body has mechanisms to deal with many of these toxins, it can't deal with all of them or an unlimited quantity. For those toxins that we cannot excrete or metabolize, the body's only answer is to store that toxin in our fat. So, the more toxic load we have, the more fat we will produce to divert and trap the toxin as a means of self-protection. As a further measure, the body will significantly resist attempts to mobilize this toxin-laden fat, making fat reduction especially challenging.

Beyond the increase in protective fat storage, the above toxins also damage our mitochondria—the “powerhouses” of our individual cells that produce the energy for every biological function. As our mitochondria become more poisoned and inefficient, we produce progressively less energy, experience “unexplained” fatigue, and experience a complex of other symptoms such as brain fog, diminished digestive ability, diffuse and non-specific pain without an identifiable cause, reduced healing ability, diminished hair growth, and once again, the inability to lose weight.

Intestinal Health

The digestive tract is our primary interface with our external environment; it performs the complex job of breaking down the foods we ingest into usable nutrients. At the same time, it also provides protection from harmful elements that should not get into the blood stream. In fact, 70% of our immune capacity is housed within our digestive system.

There are trillions of bacteria in our intestines that aid both in protecting the intestinal lining, and in digesting many foods. Unfortunately, these “good” bacteria can be killed by the antibiotics we use to treat many illnesses, and as a result, unhealthy bacteria can grow in their place. These “bad” bacteria can prevent proper digestion, so many of the nutrients that are needed (especially those that are “co-factors” for proper metabolism and weight loss) are not absorbed properly. In addition to poor nutrient absorption, these bad bacteria can damage the lining of the intestines, creating tiny cracks that can allow larger than normal food particles to enter blood stream and trigger an immune response. Eventually, the body will begin to see these food particles as “invaders” and mount an even greater immune response to their presence, leading to food “allergies.” These allergies, which are different from those that cause hives and breathing difficulties, can generate constant low-grade inflammation within the body. This inflammatory response not only robs you of energy needed for other processes, but also causes stress. Further, damaged intestinal permeability can lead to many other undesirable conditions such as depression, attention deficit disorder, anxiety, memory loss, joint and muscle pain, chronic fatigue, asthma, chronic congestion and sinusitis, rashes, acne and a host of other autoimmune-related symptoms and complexes—all of which can further add to difficulties in losing weight.

Hormonal Balance

Hormone balance is critical to maintaining proper weight, and to successful weight loss. Unfortunately, our environment is full of toxins (as you already now know) and other influences that can create hormonal imbalances and hinder weight loss. We've already touched upon insulin and its role in fat storage and prevention of weight loss, and you may already be familiar with the thyroid gland and its importance, but there are other critical hormones involved as well. Estrogen, in general, tends to push the body toward weight gain and fat storage. Eating foods packaged in tin cans, drinking water from plastic bottles, and even drinking tap water (often with traces of medications and other contaminants that have leached into the water supply) can increase your body's estrogenic load (regardless of one's gender). Additionally, peri- and postmenopausal women can become estrogen “dominant”, with lower relative amounts of testosterone and an increased propensity to gain weight. Unfortunately, estrogen and weight gain have somewhat of a “co-dependent” relationship: excess fat actually produces estrogen, as noted above. So, as one's body fat increases, in part due to the presence of excess estrogen, excess fat produces even more estrogen, and the cycle continues.

Testosterone, which aids in building muscle mass, helps support an elevated level of fat metabolism and lowers insulin levels, naturally decreases with andropause (the male equivalent to menopause), as well as with decreased exercise. Men who spend significant time in all-female environments will tend to experience a decrease in testosterone much earlier and faster than those who do not, as testosterone production is influenced by the presence of testosterone in the environment. As testosterone levels decrease, insulin levels rise, again leading to an increase in fat storage. And since muscle is very metabolically active, as testosterone decreases, it becomes increasingly difficult to maintain muscle mass and metabolic rate will decrease in step with muscle loss. Additionally, as metabolic rate decreases, abdominal fat deposits will increase, which again, increases estrogen production and helps promote fat.

Pregnenolone is another important hormone for weight loss, as it is a precursor for other sex hormones and DHEA (which then gets converted to testosterone). Not only does pregnenolone decrease naturally with age, but the natural pathways for producing pregnenolone are blocked by consuming too many saturated and trans-fats. Reduced production of pregnenolone results in reduced DHEA and in turn reduced testosterone—which means lower levels of fat burning.

Sleep

The body's primary time for releasing growth hormone (HGH) is at night, during sleep, which means that if you're not getting the proper amount and quality of sleep, you're likely not getting the proper levels of growth hormone in your system. While one might assume that growth hormone is important only for the young, in reality it is a vital hormone that impacts health across your entire life as a primary enabler of tissue repair and growth. Deficiencies in growth hormone have been linked to significantly increased risk of developing chronic heart disease and heart failure, hypertension, osteoporosis, prostate and cervical cancer, high cholesterol, glucose intolerance and metabolic syndrome, neurological degeneration, obesity and weight gain. So, lack of growth hormone is a serious health threat and a MAJOR barrier to successful weight loss.

The substance melatonin is also best regulated by healthy sleep, as it is only produced in darkness. Many think of melatonin as just a “sleep” hormone, but it is actually one of the most powerful free-radical scavengers in our bodies, and is highly protective against inflammation while also protecting our mitochondria from damage. It can help inhibit tumor growth, support the immune system, protect our brain neurons, and most importantly for weight loss, can keep insulin at youthful levels and help combat abdominal visceral fat.

In addition to hormone production, sleep levels also have the sometimes more immediately obvious impact on your state of mind and neurotransmitter levels. As your brain becomes more fatigued, your natural balance of neurotransmitters is upset, and you can become more prone to depression and anxiety, stresses that can in turn lead to metabolic syndrome.

Finally, if you are already overweight or obese, sleep can be disturbed by sleep apnea (which sometimes presents with snoring from soft palate vibration, but is NOT the same thing as apnea). With apnea, periods of arrested breathing from airway obstruction result in a lack of oxygen and the creation of inflammatory chemicals, while also putting tremendous stress on the heart and increasing the risk of heart failure. If you find that you fall asleep easily at night, and think you are sleeping eight or more hours but are still waking up very tired, sleep apnea may be an issue to evaluate. As abdominal obesity can lead to or be caused by sleep apnea, this is another major factor to consider in resistance to weight loss.

Deficiency and Impaired Weight Loss

One needs an adequate level of both vitamin D and Calcium to be able to metabolize fat correctly and lose weight.
Researchers at the University of Minnesota found that those who had the lowest levels of vitamin D had the least success with weight loss on a restricted calorie diet, and it was found that abdominal fat loss was hampered more than loss in any other body area.
Research published in March of 2000 from the University of Southern California found that lower levels of vitamin D were linked to extra accumulations of fat in the muscle tissue, which in turn led to decreased strength.

Underlying Metabolic Complications from Deficiency

When the body is deficient in either vitamin D or calcium, there is an increase in the enzyme fatty acid synthase. Fatty acid synthase is used to convert calories (specifically glucose) into fat. Having a lack of vitamin D causes more calories to be stored as body fat, particularly in the abdominal region, because it is vitamin D's job to put the brakes on this particular fat production process.

Vitamin D related weight management issues are further compounded by the body's method of creating and storing vitamin D. It has been discovered that obese people make 55% less vitamin D when exposed to the same amount of sunlight as people within a normal BMI range—which means those who are overweight will also tend to gain weight more easily. Further, as vitamin D is stored in an individual's fat cells, obese individuals may store a disproportionate amount of vitamin D, creating a perceived shortage for the body. Unfortunately, as all of this stored vitamin D isn't readily available to the body, an obese individual could be full of fat-burning vitamin D but still have low systemic or circulating Vitamin D levels.

Vitamin D has also been shown to lower leptin secretion. As we know, leptin is produced by fat cells and is intimately tied into our process of appetite creation and suppression. Without adequate vitamin D, our leptin levels can rise unchecked, in turn increasing our hunger.

While much of this discussion has focused on vitamin D, calcium is an important player as well. In addition to needing adequate calcium to inhibit the fatty-acid synthase discussed above, numerous studies have been done to show that high levels of calcium increase body temperature and metabolic rate, increase fat burning, and improve weight loss. In fact, when two groups are fed an equal amount of calories with the only difference being calcium content, the high calcium group will maintain or lose weight whereas the low calcium group does not. In fact, in a study published in 2000 in the Journal of Clinical Endocrinology and Metabolism, “Only 1000 mg of additional calcium daily can result in a 17.6 pound difference in your body weight.” In addition, this study highlighted the extreme prevalence of vitamin D deficiency in America, as 59% of the otherwise healthy women recruited for the study were vitamin D deficient (despite that the study was conducted in southern California).

Recommendations for Maintaining Vitamin D and Calcium Levels

1. Sun Exposure: It is recommended getting at least 15 minutes of sun 2-3 times per week to the face, arms, hands or back (without sun block or with SPF of less than 8), as this is the best way to maintain vitamin D levels (Sigmund CD. Regulation of renin expression and blood pressure by vitamin D(3). J Clin Invest. 2002; 110(2):155-156. (PubMed)). This is the very best way to get the most bioavailable vitamin D, with daily exposure (as weather allows) being even better.

2. Food Intake: The Institute of Medicine of the National Academy of Sciences recommends an Adequate Intake (AI) level for vitamin D, which represents the daily vitamin D intake from food that should maintain bone health and normal calcium metabolism in healthy people. Vitamin D AI for those 50 years old and younger (including pregnant and nursing women) is 200 IUs, while vitamin D AI for those 51 and older is 400 IU. The best sources of dietary vitamin D are fatty seafood, dairy, eggs, beef and cheese. The Institute of Medicine also recommends a Daily Value for calcium, namely 1000 mg for those 50 years old and younger (including pregnant and nursing women), and 1200 mg for those over age 50. The best sources of calcium are dark leafy greens and dairy products. Keep in mind that, especially for vitamin D, these are generally minimum daily values for people who are otherwise healthy or in fairly good shape. Many individuals who are overweight or obese will use more vitamin D than this, and will obtain optimum levels only with more significant supplementation.

3. Supplementation: For those attempting to lose weight, supplementation of vitamin D is appropriate to help eliminate the problem of fat loss resistance due to vitamin D deficiency. This is best achieved through therapeutic dosing as provided in the Loss phase of a program as described herein.

CLA: a Key Factor in Weight Loss & Fat Burning

CLA, or Conjugated Linoleic Acid, is a naturally occurring fatty acid found in meat and dairy products, most notably in beef, lamb, cheese, and whole milk; the food source with the highest level of CLA is butter. Ironically, the shift away from real, whole foods and the move to margarine, butter substitutes, and powdered or skim milk has actually been hurting our ability to lose fat.

It takes fat to burn fat, and CLA is one example of this principle. Over the last decade, multiple studies have been performed in which the group treated with CLA consistently loses more body fat than the non-treated group. The total weight loss with CLA in these studies is generally quite low, but the remarkable thing is, the CLA users lose weight without any calorie restriction. When CLA is combined with a low calorie, scientifically designed, anti-inflammatory diet, the results can be remarkable as it significantly boosts the body's fat burning potential.

Though the body's metabolic processes are incredibly complex, CLA has been shown to:
Block entry of fat into existing fat cells, preventing those cells from getting bigger
Shuttle fat into muscle cells for energy, helping them to grow to increase lean muscle mass, which further boosts metabolic rate
Interact with fat-sensitive hormones lipase and leptin, helping to modulate fat-burning effects and hunger response
Enhance insulin sensitivity, reducing insulin spikes needed to drive sugar into cells for fuel, and thereby reducing the messaging to the body to store fat (insulin is a “pro-storage” hormone)
Suppress arachidonic acid, which is an inflammatory compound in the body

It is very difficult to get CLA from today's food sources because of the rapid, unnatural way that cattle are typically fed. For example, in 1963, the percentage of CLA in whole milk was as high as 2.81%. In 1992, that percentage of CLA in dairy products had dropped to barely 1%. The reason for the sharp reduction in milk CLA is due to the change in feeding patterns. Cows that eat natural grass produce large amounts of CLA, but very few cattle in the U.S. are allowed to graze on grass naturally in a pasture. Today's “efficient” feeding methods rely more on hay, hormones, and antibiotics.

The best way to get the most CLA possible from one's diet is to eat free-range, grass or pasture fed meats. However, due to the unique restrictions of the program described herein (portion size, total caloric intake, and the relative lack of total fat), it is very difficult to consume enough CLA while on a program as described herein. This is why a program embodying principles of the present invention advantageously, yet optionally, includes NT7 as part of the program.

Weight-loss & body composition benefits of CLA, according to recent studies, include:
In a study of 180 people split into 3 groups, 2 groups took slightly different amounts of CLA for 1 year while the third group took none. All 3 groups were instructed to make no changes in diet or lifestyle. Both CLA groups lost a small amount of weight, but most importantly they lost 7% to 9% body fat, depending upon the dose of CLA, while the non-CLA group had no change. Additionally, both CLA groups had improvements in muscle mass, while the untreated group did not—American Journal of Clinical Nutrition, June 2004
In a follow-up of the aforementioned study, the original untreated group then took CLA for 1 year but continued with their typical diet and activity. Without any caloric restriction, this group also noticed similar improvements as the first CLA treated group. The members of the original treatment group who continued taking CLA for this second year maintained their original body fat loss—Journal of Nutrition, April 2005
A study of 25 men with significant abdominal obesity looked at the effect of supplementing with CLA for 4 weeks. The 14 men who received CLA lost an average of 1.4 cm in waist circumference after 4 weeks, again with no changes in diet or lifestyle—The International Journal of Obesity, 2001
In a study group of 60, CLA preserved muscle mass while reducing body fat, and study participants lost an average of 6 pounds—The Journal of Nutrition, December 2000
At a study in Ohio State University, diabetics who added CLA to their diet (compared to those who took a “sham” treatment of safflower oil) had lower body mass as well as lower blood sugar levels at the end of the 8 week study. The CLA group also had a 5 fold decrease in blood glucose levels compared to the safflower oil group, and showed a decrease in Leptin levels as well—Journal of Nutrition, January 2003
A group of 81 post-menopausal women were randomized to receive a CLA mixture or olive oil. At the end of the 16 week trial, the CLA group showed a 4% decrease in overall fat mass and a 7% decrease in lower body fat mass that was not obtained in the olive oil group—Journal of Nutrition, July 2009

Studies suggest that CLA is beneficial in protecting the body against:
Cancer: Animal studies show that as little as 0.5 percent CLA in your diet could reduce tumors by over 50 percent, including the following types of cancer: breast; colorectal; lung; skin; stomach;
Cardiovascular disease
High blood pressure
High Cholesterol and triglycerides
Osteoporosis
Inflammation
Immune system invaders
Food-induced allergic reactions
Insulin resistance: CLA's actions actually mimic the effect of synthetic diabetic drugs. Testing on mice with type 2 diabetes have shown CLA to improve insulin action and reduce circulating glucose. Early results from human trials are just as positive when consuming CLA for longer than eight weeks.

Some people experience short-term increases in LDL cholesterol, lipoprotein, white blood cells, and platelets, all indicators of inflammation. It is still unclear if these are by-products of increased fat burning. Some of these values return to normal with longer (2 years) supplementation, while others stay slightly elevated. Because of this, responsible supplementation with CLA occurs in an on-again, off-again pattern, generally consisting of 3 months on, followed by 1 or more months off.

Programs embodying principles of the present invention optionally incorporate NT7 in the Loss stage of the program.

All fat is not created equal. While it s true that many fats have negative effects on our health (and waistlines), consuming good fat is key to our survival. That's because fats are a necessary component of the cell membrane that surrounds every single cell in our body, making the fat in our diet essential to supporting our structure and ensuring that the body's key processes function properly. Essential Fatty Acids, or EFAs, are the good polyunsaturated fats. Because they are not produced by our bodies, we must get them from our diet. In order to maintain good health, some amount of EFAs must be consumed on a daily basis. That's because essential fats produce substances called Eicosanoids that exist in every human cell and serve many functions, including managing blood pressure, supporting circulation, and regulating pain. There are two types of essential fats: Omega-3s and Omega-6s. Our bodies' metabolic processes are dependant upon a balanced intake of these fats. For optimum health and performance, the ideal ratio of Omega-6s to Omega-3s is 3:1. Unfortunately, Omega-6s are found in abundance in vegetable oils, processed foods, and meats, and because of this, the typical American consumes a ratio that is closer to 15:1 or even 20:1. The relative deficiency of Omega-3 EFAs may be particularly harmful because they are the most potent anti-inflammatory substances available.

The most abundant supply of Omega-3 EFAs is found in fish oil which, as its name implies, is taken from the tissue of oily fish. Because these tend to be cold water fish, they often have high levels of heavy metals (especially mercury) and other poisons in their system. Actually eating the amount of fish we need to reach the proper balance of 6 to 3 Omegas would expose us to an inordinately high amount of toxic substances, so the best way to reap the benefits of fish oil is by adding high quality fish oil supplements to our daily diet.

Omega 3 EFA supplements have been show to have the following benefits:
anti-inflammatory
improve insulin secretion and insulin sensitivity
reduce triglycderide level
increase level of HDL cholesterol
anti-thrombotic (helps reduce the chance of developing clots)
reduce the chance of sudden death from arrhythmia, heart attack, and cardiovascular disease
provide small reductions in blood pressure and heart rate
improve arterial elasticity
decrease blood viscosity
improvement in skin health and elasticity
improvement in mood and memory, with a decrease in anxiety and anger
improvement in overall skin health, including tone, texture and signs of aging

In fact, Omega-3 EFAs have been found to be so beneficial, the American Heart Association recommends that people with heart disease consume a minimum of 1 gram of EPA/DHA per day (the specific type of Omega-3 EFAs found in fish oil) for individuals with heart disease, while those with high triglycerides should consume 2 to 4 grams per day.

Key factors in choosing a fish oil include:

Purity—it should be free of mercury, lead, and other toxins

Freshness—minimizing the amount of oxidation (this happens over time) ensures no fishy taste

Taste—fishy smell or taste is a sign of a poorly manufactured product

Triglyceride form—allows the oil to be absorbed optimally

Third-party testing—a manufacturer should have enough confidence in their product to submit it to a separate analysis company to determine strength and purity

Sustainability—it is obtained in an environmentally responsible way that protects the fish stock of the oceans

A program embodying principles of the present invention advantageously provides a high quality fish oil supplementation to help the body function at its peak during this time of rapid fat burning, one which meets all of the above criteria, and is optionally obtained from regulated stocks of arctic cod.

By adding high doses of quality fish oil product throughout the length of the program, a participant will enjoy all the benefits listed above, along with an improved ability to burn through stored fat because of the decrease in whole body inflammation and the improved regulation of blood insulin and glucose levels.

Caffeine and Weight Loss

If one buys a commercially available weight loss supplement, there's a very good chance that it has some level of caffeine in it. Caffeine has been used for decades as a stimulant and promoted as a weight loss aid, the thought being that it somehow “revs up” the metabolism and allows one to burn fat and lose weight faster and more efficiently. While there may be some small bit of truth to these thoughts for the well conditioned athlete who uses caffeine occasionally in relation to their workouts to spike intensity levels, for the most part, caffeine actually blocks one's ability to burn fat and lose weight in a number of different ways.
Caffeine exerts its stimulating effect in part by blocking a substance called adenosine. Adenosine is intimately tied in with the circadian rhythm cycle, and helps govern sleep. By inhibiting sleep, one continues to function and work even when the body is tired. Sleep is important for weight loss on multiple levels.
Adenosine also causes blood vessels to relax. When its action is blocked, blood vessels constrict. When they constrict, it is more difficult to carry nutrients to the cells, as well as to carry waste products and fat away from the cells. This makes clearance of fat more difficult, even if it's being broken down effectively.
Caffeine exerts a direct effect on the adrenal glands, forcing them to produce more Cortisol at a time when they normally would be at rest. This has multiple effects. First, flooding the body with Cortisol, the “stress” hormone, may make one feel better in the moment due to its soothing, comforting effects on the mind. However, long term exposure to excess Cortisol leads to increased fat deposition and arterial inflammation, setting one up for not only weight gain, but also for a higher chance of cardiovascular disease. As the adrenal glands get called upon to repeatedly release Cortisol at times when it's not necessary, they begin to get worn out and have less ability to secrete Cortisol when it's really needed, such as in the morning to help with awakening. This leads to a progressively worsening fatigue and an increasingly poor ability to deal with stressful situations, often leading to more caffeine ingestion to help cope.
Caffeine helps contribute to and speeds the development of insulin resistance, the precursor to diabetes and a major reason for fat deposition, weight gain, and increase in abdominal girth.
Caffeine prevents proper absorption of and/or increases the secretion of the micronutrients vitamin C, magnesium, potassium, zinc, and all the B vitamins. By interfering with their absorption or increasing elimination, chronic caffeine consumption can deplete all of these nutrients over time, leading to fatigue, poor exercise recovery, poor wound healing, poor sleep and weight gain.
As all of the micronutrients listed above are necessary for the body to manufacture thyroid hormone, consistent caffeine use over time can, through that same micronutrient depletion, lead to an underfunctioning thyroid and insufficient levels of circulating thyroid hormone. Since Thyroid hormones exert a significant degree of control over metabolic rate, depletion will invariably lead to weight gain over time, as well as decreased ability to burn fat due to lack of signaling from the thyroid.
Caffeine also interferes with the absorption of calcium. Calcium, along with vitamin D, is absolutely mandatory for the proper functioning of an enzyme that allows the body to break down and remove stored fat. With a shortage of calcium, the body may be set up perfectly to break down fat stores to use as energy, but still not have the ability to do so at the rate that it might otherwise be able to do so if it had enough calcium. If the deficiency is bad enough, the body may not be able to break down fat at all because of its need to guard and use its very limited calcium stores for other things.

Most of these issues are related to chronic caffeine over-use. However, the transitory decreases in micronutrient absorption, as well as immediate increases in Cortisol production, actually occur on a daily basis with consumption of each dose of caffeine. While in the Loss stage of the program, when a significant part of the goal is to lose as much weight as possible in the allotted time of the program, every little bit counts. Caffeine use will not completely prevent weight loss, and as such programs embodying principles of the present invention do not necessarily restrict caffeine entirely. However, for anyone who is losing weight slowly, caffeine ingestion may in fact be exacerbating the issue and working against one's otherwise best efforts. In such cases, minimizing or eliminating caffeine from one's diet help create the best chance for improved results.

Seven

A recent 30,000 person study conducted by West Virginia University and published in The Journal Sleep found that the optimal amount of sleep each day is seven hours. While one might reasonably expect that getting less than seven hours of sleep would be detrimental to health, perhaps more surprising is the fact that getting MORE than seven hours of sleep is also correlated with increased health risks. The study showed that those who sleep less than seven hours, including naps, more than double their risk of cardiovascular disease—being diagnosed with angina, coronary heart disease, heart attack or stroke. Certain groups had even greater risk of cardiovascular disease with too little sleep—adults under 60 years of age had a threefold risk, and women had two and a half times the risk. While not quite as detrimental, the study also showed that those who slept nine hours or longer each day (including naps) were one and a half times more likely to develop cardiovascular disease than those sleeping seven hours, even when controlling for other major health factors. These results are in line with a major 2002 study from the University of California that first identified the link between excess sleep and increased mortality, as well as research from Columbia University and University College Medical School in London—both of which also identified a link between too little sleep and a 50% increased risk of developing diabetes within a ten year period.

The causes of increased health risk may be slightly different for the under-sleepers and over-sleepers. The human body uses sleep to repair itself, releasing hormones that fight inflammation and support cell repair. So, those with less sleep can over time become victim to inflammatory diseases such as cardiovascular disease, and to diminished metabolic functioning which in turn can cause obesity and reduced muscle mass while contributing to further hormonal imbalance. The phenomenon of higher risk from over-sleeping, while now observed in multiple studies in different countries, is not well understood. There are a number of potential explanations, ranging from circadian sleep cycle misalignment to the need for a particular level of activity and healthy stress through waking hours. No matter the cause, targeting seven hours of sleep would seem to be a priority for maintaining health. If for some reason a person is unable to remain alert or have adequate energy with seven hours sleep, there may be other issues at play.

Smoking

The effects of smoking during programs as described herein are the same as at any other time—there is a short term appetite suppression and increase in metabolic rate, at the cost of very serious (fatal) long-term consequences. While there are no comparative studies of the efficacy of dieting for smokers versus non smokers, the physiological effects that occur secondary to smoking do tend to impair some of the important processes that allow the body to eliminate fat as quickly as possible.

Impact of Smoking on Weight Loss Processes

Programs as described herein take a strong stance against toxins due to their effect on different enzymatic and metabolic processes in the body, and potential impediment to weight loss—and smoking is equally strong. Smoking contaminates your blood cells, which carry oxygen to your body, infiltrating these with carbon monoxide (poison). This makes it harder for the circulatory system to oxygenate the body and can cause you to be tired or have lower energy. Smoking also causes a general “vasoconstriction” or diminished diameter of your blood vessels, decreasing blood flow to various areas, but especially to areas where the blood vessels are smallest (including fat storage areas). If blood flow is impeded, so are the chemical signals to break down the fat and the ability to pull nutrients back into the system for fuel and elimination. Further, the carcinogens in cigarettes poison mitochondria, the little energy creators of cells, and a person needs high levels of energy to successfully maintain a rapid level of weight loss.

Quit Smoking

Obesity and smoking are the two greatest risk factors for a person's health, and so it can be particularly advantageous to discontinue smoking as well as to achieve a healthy body composition. Quitting smoking is often associated with weight gain due to the use of food as a replacement for habitual smoking, the absence of hunger suppression from nicotine, and the improvement in/return of taste, which makes eating more enjoyable. This potential for weight gain is commonly an impediment for people who would like to quit, and a reason for continuing in spite of knowledge about the negative health effects of smoking. As one is likely already working hard to maintain eating discipline during a program as described herein, and also getting a significant amount of help in suppressing appetite, quitting smoking during a program as described herein may be easier than at any other time, and every attempt should be made to use this opportunity of enhanced appetite suppression to also try to quit smoking.

When it comes to your skin, the saying “You are what you eat” often holds true, as skin issues are commonly a sign of problems that emanate from food intake and the digestive system. So it only makes sense that your skin may experience changes on a program as described herein, which can eliminate common systemic allergens such as wheat, dairy, corn, and high glycemic-index carbohydrates while cleansing the body with ample amounts of water and burning reserves of toxin-storing fats.

Low Glycemic Load and Low Allergen Diets: Reducing the Causes of Chronic Skin Conditions

Because eczema and other skin irritations can be related to internal imbalances such as food sensitivities, these are often the first types of skin issues to clear during a program as described herein. Another skin condition that has some root in diet is acne, and participants may experience an improvement in this as well. Research suggests that one potential acne-provoking factor is the consumption of certain high glycemic-index carbohydrates. In addition to causing inflammation, these foods stimulate the production of copious quantities of the blood sugar-regulating hormone insulin. Laboratory experiments show that surges of insulin in the system encourage the secretion of sebum, and may also initiate changes in the skin that tend to block the sebaceous glands that make it. It is the overproduction of sebum in the skin, often coupled with bacterial infection, that causes pimples and acne. In 2007, an Australian study tested the effects on acne of a carbohydrate restricted diet. In this study, 43 participants were assigned one of two diets: One a low glycemic load (GL) diet, of which 25% of meals came from lean protein and 45% from low glycemic index carbs; and the other a control diet heavy in high glycemicindex carbs. After 12 weeks, the total count of acne lesions in those on the low GL plan had decreased by about 24 compared to about 12 in those on the control diet. Inflammation in the skin had also decreased by about twice as much in the low GL dieters compared to the high LG dieters. Overall, the low GL diet led to a 50% decrease in the number of acne lesions.

Evidence suggests that eating lean proteins and carbs from natural sources such as fruits and vegetables while foregoing those more processed choices—as is the case with programs as described herein—makes for healthier, clearer skin.

The Short Term Impact of Detoxification and Fat Burning: Temporary Acne Flare-Ups

While there can be substantial benefits for a participant's skin in the long term from a low GL diet, this diet can also initiate a detoxification process within the body. Sometimes this initial release and subsequent clearing of toxins can cause breakouts. One major factor is the elimination of “bad” bacteria in the intestines. As this occurs, not only does this bacteria release toxins as their cells break down, but the make-up of the bacteria on the skin begins to change as well. A second major factor is the release of toxins that have been stored in the body fat that is now being burned for energy. These toxins will circulate through the body before they are ultimately eliminated. Even though these changes are for the better, the skin often responds with a temporary break-out of acne before settling into its new, clearer state—these breakouts could be anywhere on the body or the face.

The best thing to do to is to continue to be diligent about water intake and stick to the diet as closely as possible. In the meantime, acne can be treated acne with a combination of benzoyl peroxide and salicylic acid.

Improving Gut Health

Understanding Gut Bacteria

There are over 400 different bacterial species and an estimated 100 trillion bacteria residing in each human gut. Overall, there are 10 times more bacteria cells in a human gut than cells in the human body. The combined metabolic activity of gut bacteria is greater than that of the liver, an organ that is constantly filtering toxins from our blood and changing macronutrients into different useable forms to adjust to our nutritional needs. These gut bacteria perform a number of different functions, but are primarily responsible for helping us to digest food and assimilate nutrients while protecting the lining of the intestines from damage by “bad” bacteria or foods. Gut bacteria developed with humans over tens of thousands of years, and the bacteria that survived in the human intestines lives best on a hunter/gatherer type of diet of vegetables, roots, tubers, fruits and proteins.

Unfortunately, our current western diet doesn't readily support the health of critical “good” bacteria. As industrial farming and food manufacturing have shifted human diets such that grains, breads, and high carbohydrate, high sugar foods have become the predominant source of calories for a large percentage of the population, the good bacteria in our guts have not had time to adjust to this dramatic and rapid dietary change. Instead, the good bacteria get overwhelmed by the simple sugars that are ingested, and the intestines are taken over by other types of bacteria that are not as beneficial, and are in some cases harmful. These “bad” bacteria feed on the modern high sugar diet, and can be responsible for sugar cravings as well as numerous negative health effects. Note that several other factors prevalent in our modern lives can also kill good bacteria, including stress, antibiotics, alcohol and antacids, to name just a few.

Impact of Bacterial Imbalance

This shift in the balance and composition of gut bacteria is called Intestinal “dysbiosis”, and is associated with a laundry list of symptoms that are quite familiar to most Americans, including: Nausea; Belching, bloating; Heartburn; Abdominal pain; Cramping and abdominal distension; Constipation and/or diarrhea; Abdominal spasms; and Flatulence.

As good bacteria dies off, our ability to digest certain foods and extract nutrients from those foods decreases. Also, our intestinal walls have less protection and can become inflamed from repeated damage, causing a further decrease in intestinal function. This process eventually reaches beyond the intestines, affecting our entire system because of nutrient mal-absorption and the slow, microscopic leaking of undigested particles from the gut into the bloodstream where these particles were never meant to be. This can be a root cause of many systemic, allergic-type symptoms that reflect a hyper-reactivity of the immune system, including: Joint pains; Acne; Anxiety; Brain fog; Slow thinking and memory deficits; Depression; Fatigue; Unexplained fever; Frequent urination; Flu-like malaise; Muscle aches; Palpitations; Itching; Vein inflammation; Skin rashes; and Seizures.

Restoring Bacterial Balance

Programs as described herein are designed to help restore gut health by setting the stage for regrowth of normal, healthy human gut bacteria. This rebalancing process involves the “starving” of “bad” sugar-burning bacteria so that it will die off, allowing “good” bacteria to take its place as proper diet is reintroduced. Through this process, the participant's overall health improves due to reduction in systemic inflammation, improved digestion, better nutrient assimilation and elimination of cravings. As the body becomes healthier in these ways, the metabolism improves and weight loss becomes easier.

Changes in Bowel Habits Due to Bacterial Rebalancing

One potential issue that can occur during the bacterial rebalancing process is a significant change in bowel habits. As the “bad” bacteria die off there can be a period of time where some people experience significant and/or prolonged diarrhea or constipation. The onset of this change in bowel habits can occur as late as several weeks into the program, reflecting the point at which the bad bacteria are no longer able to survive and are purged—and when good bacteria are not present or repopulated in sufficient quantity to stabilize the digestive system. While this phase is temporary, the onset can be sudden, and as such it is not uncommon for some to believe they may be reacting to a specific food or component of the program. Nonetheless, bowel habits do eventually regulate again once gut bacteria comes into better balance.

Probiotics to Support Gut Health and Bowel Habits

One of the best ways to help with this temporary issue, should it occur, is to take probiotics. Probiotics are supplements containing large amounts of the healthy gut bacteria. Supplementing these bacteria at a time when the “bad” bacteria are dying off assists new healthy bacteria to repopulate the intestines more quickly, restoring more normal digestion and bowel habits. There are many probiotics commercially available, and in general a mixed-strain product that allows the participant to take in 10-20 billion CFU (colony forming units) daily, as described herein, can be advantageous. As a frame of reference, most yogurts will have ½ a billion to 2 billion CFU at most per serving (a very small fraction of what a participant needs), so supplementation is required to have a meaningful impact on intestinal bacteria repopulation—whether eating normally or on a restricted diet such as described herein.

Food and Cosmetic Toxins

Organic Food and Cosmetics for Improved Weight Loss

Toxins in the foods we eat and on the foods we eat—especially in the form of pesticides—can impact the weight loss process as these build up in our system and decrease our ability to efficiently and appropriately respond to the nutrition that we consume. In addition, there is now amassed substantial evidence that the skin has numerous pathways by which chemicals in cosmetics and topical products can be absorbed and become toxic in our system, to similar or even worse effect than the contaminates in our food. The combined effect of such toxins is to make it harder to lose weight, while simultaneously contributing to or causing abnormal weight gain.

SLIM Stage

During the Slim stage of a program embodying principles of the present invention, a participant will consume 1200 calories each day. During this time, the participant is asked to continue to eat the same kinds of food that they have been eating throughout the program, but in addition to eating greater amounts of food, the participant may also add the more fatty fishes (such as salmon), and a small amount of oil for cooking and flavoring purposes. The participant may begin exercising more vigorously again, but it is advantageous when the participant waits until they have been on the Slim stage diet for about 3 days before beginning an exercise program of any significance. The SLIM stage advantageously is about 30 days long, although shorter and longer periods can be used.

For Those Intending to Complete Additional Cycles

If the first round of a program as described herein was not enough to reach a desired target weight, a second cycle of a program as described herein can be started 30 days after the SLIM stage. Alternatively, the participant can proceed to the next stage.

Breakfast:

1. One serving of lean meat, beans, dairy, fish or eggs from the lists as described herein in the designated breakfast portion.

2. One serving of fruit from the lists as described herein in the designated portion.

Lunch:

1. One serving of lean meat, fish, beans, dairy or eggs from the lists as described herein in the designated lunch portion.

2. One serving of vegetables OR leaf greens from the lists as described herein in the designated portion.

Snack: One ounce of nuts, from the list as described herein, or 3.5 ounces of avocado or 4 ounces of olives (pitted, no filling, Kalamata olives not allowed), daily.

Dinner:

1. One serving of lean meat, fish, beans, dairy or eggs from the lists as described herein in the designated dinner portion.

2. One serving of vegetables OR leaf greens from the lists as described herein in the designated portion.

3. One serving of fruit from the lists as described herein in the designated portion.

Seasoning, beverages, sweeteners, measuring, and cooking as described above.

Calories by Food Category

| Food Category | Breakfast | Lunch | Dinner | Snack | Sub-total |
|---|---|---|---|---|---|
| Protein | 130 | 185 | 185 | 0 | 500 |
| Vegetable or Green | 0 | 60 | 60 | 0 | 120 |
| Fruits | 70 | 0 | 70 | 0 | 140 |
| Oil & Seasoning | 30 | 50 | 50 | 0 | 130 |
| NTx (w/bev.) | 45 | 45 | 0 | 0 | 90 |
| Snack | 0 | 0 | 0 | 180 | 180 |
| Sub-total | 155 | 420 | 425 | 180 | |
| Total | | | | | 1160 |

Nutritional Therapeutics Guidelines

Across the course of a program as described herein, the participant will be using a combination of pharmaceutical grade therapies to support a weight loss plan. The nutritional therapeutics (NTx) are taken as indicated below:

AM Pill Pack:

NT4—2 capsules

NT6—2 capsules

NT15—2 capsules

NT13—1 capsule

Mid-Day Pill Pack:

NT4—2 capsules

NT6—2 capsules

NT15—2 capsules

PM Pill Pack:

NT17™—7 capsules

NT18—1 capsule

Key Therapies Regimen

Insulin and Glucose Stabilization: NT4

Endocrine Support: NT4

Digestive Health: NT17, NT18

Good Cholesterol and Anti-Inflammatory Support: NT15

Fat Mobilization Support: NT13

Energy Support: NT6

Cortisol Stabilization: NT4

NT17

Provides therapeutic levels of L-glutamine, N-acetyl-glucosamine, MSM, DGL, slippery elm, marshmallow, chamomile, okra, TOA-free cat's claw, quercitin, and mucin for comprehensive support of optimal gastrointestinal health. Prunus and citrus pectin are included to aid regularity.

Serving Size: 7 Capsules

Amount Per Serving

L-Glutamine 1500 mg

N-Acetyl Glucosamine 2.0 g

Citrus Pectin 1000 mg

Deglycyrrhizinated Licorice 400 mg (DGL) (Glycyrrhiza glabra)

Aloe Vera (Leaf Extract 200:1) 300 mg

Slippery Elm (bark) (Ulmus pulmila) 200 mg

Mucin 200 mg

Marshmallow (root) (Althea officinalis) 100 mg

Chamomile (Matricaria chamomilla) 100 mg

Okra (pod) (Hibiscus esculentus) 100 mg

Cat's Claw (TOA free) (bark) (Uncaria tomentosa) 100 mg

Methylsulfonylmethane (MSM) 100 mg

Quercetin 100 mg

Prunus (Concentrate) 100 mg

PepZin GI (Zinc-Carnosine) 75 mg

Other Ingredients: Microcrystalline cellulose, magnesium stearate.

NT18

Delivers *15 billion organisms per dose in a caplet form—and uses an advanced delivery system to ensure delivery of the highest number of live organisms to the intestinal tract.

Probiotic Blend 15 Billion cfu

(L. acidophilus)

(B. bifidum)

(L. casei)

(L. rhamnosus)

(L. reuteri)

(B. breve)

(B. longum)

(S. thermophilus)

Other Ingredients: Hydroxypropyl methylcellulose, pectin, microcrystalline cellulose, sodium carbonate, silica dioxide, stearic acid, turmeric.

*viable organisms at time of manufacture

STABLE WEIGHT Stage

A Stable Weight stage embodying principles of the present invention advantageously extends over a 90-day period, although somewhat shorter or longer periods can be used. At about the 8^{th} day, a first daily grains portion can be added; at about the 15^{th} day, a second daily grains portion can be added; at about the 22^{nd} day, a third daily grains portion can be added; at about the 29^{th} day, substitutions are allowed.

The recent Great Recession of 2008 and subsequent retrenching of American business and consumers provide an apt analogy for the back-to-basics mentality that necessarily characterizes a participant of a program as described herein. After perhaps many years of eating too much or without adequate thoughtfulness about the consequences of exactly what one was eating, transition to a new, healthier approach to eating will require adopting a new perspective, grounded in the reality of what it means—and what it takes—to eat well. As will be seen, the physical quantity or volume of food allowed on this stage of a program as described herein is substantial (especially in comparison to the low calories phase), but the composition of the food continues to be a matter of critical importance. The stage has been set for the Stable Weight stage by the lessons from the previous stages, which eliminated the chemicals, sugars, and processed foods that are so prevalent in most diets. In response to these changes, a participant likely experienced not only substantial weight loss, but also a sense of well-being and control not felt in years—this is due at least in part to the return to eating whole, simple foods in appropriate quantities.

One objective of a participant is to make the new eating habits stick permanently, albeit with larger quantities and eventually a broader range of healthy foods. In this regard, much of what is learned during the prior stages becomes the basis for a new way of life—with the low calorie phase NOT just some temporary period after which the participant returns to the same old approach to eating. While the Stable Weight stage is very manageable, it nonetheless also has challenges, as does the longer term adoption of healthy eating habits. Specifically, the New Normal for the participant's dietary habits is going to be about:

1. Basic, Simple Foods—Rich in nutrients and fiber, but low in chemicals, additives, pesticides or anything artificial.

2. Worthwhile Inconvenience—Healthy food is, sadly, very hard to find without planning, and as such it requires real, concerted and sustained effort to eat the right things each day. This entails energy and dedication, as the world is not organized to make healthy eating convenient or inexpensive. But a recognition that most of the food industry is working at cross-purposes with you when it comes to your health is critical for the participant's own long-term success.

3. Living with Greater Awareness—The benefits experienced during a program as described herein can be extended into one's everyday eating by continuing vigilance and paying attention to the food and beverages consumed—and with a fundamental recognition that most manufactured food poses risks that have contributed to the obesity epidemic faced in our country.

4. Moderation—Especially coming off of a period of reduced food intake, one needs to mitigate the risk of a “pendulum swing” or binge through planning and proper mindset.

In the case of participants for whom weight gain was a very gradual process, over many years and in small amounts each year, there is good likelihood that a modest amount of care and increased awareness will allow one to maintain their newly reduced body weight with relative ease through both diet and exercise. Even for those participants who gained substantially in a short period of time, but for whom diet discipline had not previously been a problem, the ability to resume control over portions and food types may not be especially difficult—particularly after a “reset” of habits and taste during a program as described herein.

There are many participants, however, who have had a different or more acute struggle with food over time, and for whom self-control has been a long-standing issue such that a real fear may exist about the ability to manage one's own diet without quickly regaining weight. It can therefore be considered the framework of detox and rehabilitation as a parallel to the weight loss process and return to independent eating.

The Concept of Food Addiction

There is some debate in the medical community as to whether the term “food addiction” is appropriate for describing the need that many have to eat without regard to any true caloric requirement. Nonetheless, there is substantial and growing research suggesting that sugar in particular is perhaps one of the most addictive substances in the world, and that the brain response to food (its availability as well as its consumption) can mirror the response to certain drug categories. Specifically, the consumption of sugar is believed to cause the brain to release opioids that increase the cravings for that substance. In fact, even the sight of foods with sugar is believed to elicit the release of opioids and cause intense cravings—much like with other types of addictions. Given the parallel between addiction to certain drugs and the brain mechanisms and response to food, it is worthwhile to consider the challenges of changing one's dietary habits as similar to the challenges of eliminating and managing against addictions.

Typically there are several phases of addiction treatment—with the first being a detoxification phase. Whether gradual or all-at-once, this is a period when the body is purposely denied a substance so that it is no longer subject to the effects of that substance. The next phase is rehabilitation, where new habits are practiced, and education is provided to give perspective and preparation to those wishing to avoid a return to addiction. Finally is the maintenance phase that is ongoing, in which those habits and learning are implemented fully, with the recognition that continuous effort and vigilance must be maintained in order to avoid a return to the habits of a previous addiction.

In keeping with the addiction treatment analogy, one can think about programs as described herein as a program structured to:
manage participants through stages of detoxification and retraining
set participants on a course for long-term weight management success

In doing so, the objectives and risks of each of these stages becomes clearer—and the long term importance of getting each of these stages right can also be more evident. A major challenge to maintaining discipline with a healthy diet is that, at least for now, the world seemingly conspires to make one fail by putting into much of our food the additives (such as sugars and artificial sweeteners) that one is now trying so hard to avoid.

One mental trick to avoid sugars, chemically enhanced and processed food is to think of oneself as having a debilitating addiction, or even an acute allergy to those foods—and to convince oneself of the dire consequences of partaking in these. Since we know that the immediate fear response is often the most powerful way to change behavior, a participant might want to use this to help cement the change to their eating. Armed with an understanding of the types of foods to eat—and those to avoid—a participant can adopt healthy eating, as outlined herein. One aspect of the Stable Weight stage is to take advantage of the participant's newly “detoxed” system and new eating pattern, and to further ingrain the healthy eating habits that can carry the participant forward.

During the Stable Weight stage, it can be important to long term success to continue with these eating habits while allowing ingestion of other low glycemic-index carbohydrates. Not all carbohydrates are evil, and they certainly have some benefits for the body when eaten judiciously (for instance, carbs before a work-out help improve performance and recovery, as well as post exercise muscle growth).

Inverting the Food Pyramid

The USDA food pyramid—it is perhaps the largest and costliest mistake in human history. The extent of this mistake can be measured in human lives (mortality), quality of life (morbidity), and monetary cost (health care). While the food pyramid was created in part to facilitate the ready and widespread availability of food and the corresponding development of mass food production, the result has been disastrous not only for the US, but also for countries to which the US has exported its food and production model—along with the resultant obesity levels. This model among other things systematically eliminates fiber from human diets while it systematically inflates or introduces unhealthy ingredients. Most importantly, it gets basic nutrition wrong.

The Key Food Pyramid Error—Carbohydrate Intake

The suggestion of consuming most of our calories in grains and carbohydrates is counter to millennia of evolution. The ingestion of large quantities of food that our body is not designed to handle has led our descent into obesity, heart disease, stroke, dementia, allergies and autoimmune diseases. Like all animals, humans evolved to exist in the most efficient way possible given the conditions and the resources available. We developed over a million and more years surviving on vegetables, roots, tubers, fruit and meat (from insects, fish and mammals). Grains were an almost non-existent dietary component until about 10,000 years ago, and even then were not a significant portion of the human diet. Our bodies developed to operate at peak efficiency with very low carbohydrate, high fiber foods that typically required a high level of processing (through digestion) to extract nutrition or sugars, with humans subsisting on a significant proportion of fiber and protein.

The introduction of large quantities of low/no fiber, high carbohydrate foods has meant access to more “on-demand” energy than ever before. Not only do our bodies not have to work for this energy, but carbohydrates are continuously available in excess. Add to this the fact that our bodies are designed to survive in times of famine with an orientation toward energy storage whenever excess calories are presented, and the stage is set for a metabolic disaster.

Carbohydrates and Metabolism

Carbohydrates cause an increase in insulin production, which in turn allows those sugars to enter your cells to provide energy. Insulin is a necessary substance, but we weren't meant to have large amounts of it circulating through our bloodstream constantly. When our cells are bathed in it they become resistant, requiring more and more insulin to drive the same effect. Insulin, however, is a pro fat storage hormone, signaling the body to store as much energy as possible in expectation of future food scarcity. Even though for most of us, famine has never and will never come, the body has no way to know that it should shut off storage, and in the face of abundance, it continues to build fat.

Unfortunately, insulin isn't just a pro-storage hormone, it's also inflammatory. In small quantities and for short periods this is a good thing, as inflammation helps the body to heal and repair. However, constant insulin production becomes a driver of constant inflammation, leading to a break-down in metabolic efficiency and a decreased ability to lose weight, along with a significant increase in cancers. our body responds to its state of constant inflammation with yet another, very powerful anti-inflammatory hormone—cortisol, which is produced by your adrenal glands and was meant to help with short term, stressful situations. The constant consumption of carbohydrates and production of insulin means we are now also subject to excess cortisol over extended periods. Such exposure contributes further to increased fat deposits, as well as to decreased concentration, decreased immunity, tissue breakdown, cardiac disease, dementia, and much more.

The Good News

Through modification of the participant's diet and short periods of regular exercise, the participant can send this cascade of unhealthy metabolic reactions in reverse, and begin healing the body and losing weight. The key is to address the root cause of weight gain by inverting the food pyramid. Carbohydrates should be at the low end of your volumetric and caloric consumption, and simple, high-glycemic index carbohydrates should be reserved for “treat” status. A good dietary ratio for the long term is 40% protein, 35% complex carbohydrates and 25% good fats (mono or polyunsaturated fats with an Omega 6 to Omega 3 ratio of no higher than 6:1, and ideally 4:1). If 25% of calories from fat seems high, realize that the Inuit people have a diet that at times can reach 80% to 85% fat, yet have very few of the chronic disease issues that plague the U.S. population. In spite of our trained and typically misguided obsession with avoiding all dietary fat, fat in fact plays an important role in a balanced diet.

New Food Pyramid

The base of the participant's pyramid should be vegetables. The participant should try to eat at least one of each different color of vegetable every day—green, yellow, orange, red, purple. On a typical plate, ½ should ideally be a vegetable serving, ¼ should be a protein serving, and ¼ can be a complex carbohydrate, ideally from the low glycemic load list described herein.

Just above vegetables on the food pyramid should be protein. Continue to choose, as much as possible, organic, free range or wild caught protein. Now that the participant is in the long-term phase of a healthy eating program, the participant can feel free to eat liberally from the cold water fatty fishes, as the omega 3 fats present in their meat is an excellent anti-inflammatory substance and will help keep a healthy balance of fats. Additionally, high levels of omega-3 (also found in much higher levels in free-range beef and bison than in their regular counterparts) help the body with weight loss.

Next up on your food pyramid should be fruits and nuts. Try to get fruit servings from the real fruit, not fruit juices or dried or canned fruit. These latter options strip out all the fiber that is naturally present in the real whole fruit, and leave the body with a significant sugar load to process. When sugar is ingested with fiber, as would be the case in eating whole fruit, the absorption of the sugar is slowed greatly, thereby decreasing the resultant insulin spike and minimizing inflammation and fat storage, while also getting the fiber benefits of reduced incidence of colon cancer. For nuts, one to two handfuls a day is ideal, with walnuts, almonds, and pistachio's having the highest known benefits. Try to avoid eating too many peanuts, as the nutrition profile is not as healthy nor is the harvesting and processing that many peanuts go through to get to the market.

At the very top of the food pyramid should be breads, grains, pastas, cereals, etc. These are the carbohydrates that you should try to ingest the least, because even in their complex form they present more of a carbohydrate/sugar load than your body is designed to have. That doesn't mean they need to be avoided altogether, but that they need to be eaten with care and moderation. As much as possible, try to choose foods from this group that are high in fiber and part of the low glycemic index, low glycemic load group. Especially when eating simple carbohydrates from this group, try to eat another protein or fiber source at the same time to slow the sugar impact and blunt the insulin response. At the top of this group are also included foods such as ice-cream, candies, pies, cakes, etc. A once per week “treat night” is a good strategy for limiting these items.

Food Volume and the Shrinking Stomach Myth

While you will commonly hear people assert that their “stomach has shrunk” due to a period of restricted eating or change in dietary pattern, the reality is that the human stomach cannot actually permanently shrink—at least not without surgery. So what explains this commonly held misperception? According to the American Journal of Clinical Nutrition, a low calorie diet of four weeks or more increases a person's sensitivity to the satiety homone (leptin), or decreases one's resistance to this hormone. This means that one feels “full” more quickly or with a lesser volume of foods. The hormone now signals to the brain that enough has been eaten at a food volume that is often substantially less than one would have previously required to feel satisfied. Additionally, there are nerves that take the message of expansion and contraction of the stomach back to the brain. Over the course of a few weeks, if the amount of food that is consistently being put into the stomach is lower so that the stomach never expands as much as it did with larger meals, the brain will “re-set” in its interpretation of what feels full. Essentially, the brain will interpret a smaller volume of food as being just as filling as a larger volume used to feel.

Satiety

This phenomenon of lesser eating capacity can make Stable Weight seem like even more than the increase of food intake that it really is. In fact, it is not uncommon for participants of programs as described herein to be unable to eat the entire daily calories as specified, at least in the initial week or two. The short term benefit is typically additional weight loss. Note that as one's body is constantly adapting, one will eventually become accustomed again to the greater level of food intake.

Stable Weight Background and Principles

Guiding Principle: As much as possible, eat only real food. This means food that is not processed, does not have chemicals or artificial sweeteners added, and generally would spoil after a week. While one's food may be packaged and labeled by necessity, these labels should have few and very simple ingredients (since this food is not “engineered”)—not long lists of substances that are unpronounceable, and would have been generally unrecognizable to someone just 100 years ago.

Calorie Level and Activity Requirement

The Stable Weight dietary guidelines are designed to correspond with the average daily base metabolic ranges for women (1300-1500 calories) and men (1500-1700). The allowed calorie levels for women (approximately 1,800) and men (approximately 2,000) presume that participants continue their walking activity (or equivalent) daily. If one is unable to complete the required 10,000 steps in any day (within 2,000 steps), the participant should eliminate one fruit portion and one grain portion from the diet on that day (ideally one portion from two separate meals if possible).

Adjustments to Calorie Level and Portion Size

Portion Reductions: A small percentage of people may have a base metabolic rate that is substantially lower than average—as low as 1,100 calories per day. This group is most likely to include women with lower quality muscle or lower amounts of muscle mass. This lower metabolic rate may nonetheless be offset through activity level (short term) and through increases in muscle mass (long term). In these less common cases, it could be necessary to reduce portion sizes or to eliminate certain portions to adjust for lower metabolic rate, particularly for those who are inactive and do not regularly engage in resistance exercise to improve muscle quality and increase muscle mass. If one is correctly following the Stable Weight guidelines described herein, but notice that they have gained and retained weight during the Stable Weight stage (look at a 2-week period for evaluation to allow for normal fluctuations), food portions can be modified so that the participant can maintain a balanced food intake without hunger or additional weight gain.

Portion Increases: There may be days when calorie burning from activity is higher than the standard activity protocol (10,000 steps or equivalent). In those cases when the participant exceeds the base daily activity requirement, they may eat additional calories safely. As a general guideline, for each 30-45 minutes of moderate to vigorous exercise (maintaining an elevated heart rate for this period) beyond the standard activity level, one may add one portion of fruit, grains or vegetables. Should the participant exercise for multiple additional 30-45 minute periods, do not add more than one additional portion to any single meal.

Stable Weight Suggestions for Balancing Food Consumption

Red Meat: Limit to 1 portion per day, 2-3 days per week (cholesterol)

Dairy: Limit to 1 portion per day, 2-3 days per week (inflammatory)

Soy: Limit to 1 portion per day, 1-2 days per week (phyto-estrogen)

Peanuts: Occasional consumption only; avoid when alternatives available (toxins)

Animal Protein: We recommend one vegetarian day per week (cholesterol)

Shifting Portions: Eating meal portions between meals as snacks is allowed (hunger management)

Combining Portions: Do not combine portions from multiple meals at one meal

(blood sugar)

High Glycemic Grains, Fruits and Vegetables: limit high Glycemic Index foods to one serving per week each, most notably—potato, corn (as vegetable), corn (as grain), millet, dates and watermelon.

Glutens: Please be careful to observe any digestive issues following consumption of grains containing gluten (noted next to grains where relevant).

Time Between Dinner and Breakfast: it is recommended to wait 11-12 hours between the final meal of the day (or any food eaten in the evening) and breakfast, an approach supported by recent longevity studies.

Night Time Eating: Eating before bed does not cause weight gain (as long as within daily calorie limits), but can cause heartburn or sleep disturbances for some. As such, carefully monitor your sleep for changes if you eat close to bed time.

The Stable Weight Dietary Guidelines allow for a substantial increase in calories—50% more for women and 66% more for men over the prior stage's Guidelines. It is best to make a transition to this increased calorie load, and dramatically expanded set of foods, sequentially across the first full month rather than doing so immediately. The objectives during this initial month of transition are to: avoid the feeling of overeating; avoid accidental overconsumption of calories; gradually incorporate additional foods for both variety and nutritional benefit; become accustomed to new portion sizes; observe changes to weight and modify activity level and/or portions if necessary; carefully monitor any physiological effect from new foods (including digestive system distress, change in sleep quality/snoring, water retention and inflammation, secondary effects of inflammation such as joint pain or allergies, headaches, concentration/focus, increases in hunger or cravings, energy level). As such, a participant following a program as described herein advantageously uses the following structure to transition across the first month of the Stable Weight stage:

WEEK 1: no grains or substitutions—follow all other the Stable Weight guidelines

Objective—Become accustomed to new portions and food options; observe impact of added foods

WEEK 2: 1 grain portion daily and no substitutions—follow all other Stable Weight guidelines

Objective—Begin to introduce grains and monitor impact on digestion, energy, cravings

WEEK 3: 2 grain portions daily and no substitutions—follow all other Stable Weight guidelines

Objective—Continue introduction of grains while monitoring impact

WEEK 4: 3 grain portions daily and no substitutions—follow all other the Stable Weight guidelines

Objective—Complete introduction of grains while monitoring impact

WEEK 5+: Follow the Stable Weight Guidelines as described herein, including Substitutions

Objective—Solidify sustainable, long-term dietary habits

Breakfast:

1. One serving of lean meat, beans, dairy, fish or eggs from the lists described herein in the designated breakfast portion.

2. One serving of fruit from the lists described herein in the designated portion.

3. One serving of grains from the lists described herein in the designated portion.

### (Starting on Day 8)

Lunch:

1. One serving of lean meat, fish, beans, dairy or eggs from the lists described herein in the designated lunch portion.

2. One serving of vegetables from the lists described herein in the designated portion.

3. One serving of leaf greens from the lists described herein in the designated portion

4. One serving of fruit from the lists described herein in the designated portion.

5. One serving of grains from the lists described herein in the designated portion.

### (Starting on Day 22)

Snack: One ounce of nuts, from the list described herein, or 3.5 ounces of avocado or 4 ounces of olives daily.

Dinner:

1. One serving of lean meat, fish, beans, dairy or eggs from the lists described herein in the designated dinner portion.

2. One serving of vegetables from the lists described herein in the designated portion.

3. One serving of leaf greens from the lists described herein in the designated portion.

4. One serving of grains from the lists described herein in the designated portion.

### (Starting on Day 15)

Seasoning, beverages, measuring, cooking and substitutions as described above.

Reduce daily food consumption by 1 Fruit serving and 1 Grains serving on days when minimum activity requirement of 10,000 steps (or equivalent) is not achieved (within 2,000 steps). One may increase consumption by 1 serving of Fruit, Grains or Vegetables for every 30-45 minutes of additional moderate to vigorous exercise (maintaining an elevated heart rate for this period); do not add more than one additional portion to any meal.

Substitutions allowing for limited consumption of Alcohol, Chocolate and Butter are permitted. Substitutions for fruit, vegetables and grain portions are permitted.

Women

| Food Category | Breakfast | Lunch | Dinner | Snack | Sub-total |
|---|---|---|---|---|---|
| Protein | 175 | 225 | 225 | 0 | 625 |
| Vegetable | 0 | 90 | 90 | 0 | 180 |
| Greens | 0 | 30 | 30 | 0 | 60 |
| Fruits | 100 | 100 | 0 | 0 | 200 |
| Grains | 120 | 120 | 120 | 0 | 360 |
| Oil & Seasoning | 60 | 60 | 60 | 0 | 180 |
| NTx (w/bev.) | 10 | 0 | 0 | 0 | 10 |
| Snack | 0 | 0 | 0 | 180 | 180 |
| Sub-total | 465 | 625 | 525 | 180 | |
| Total | | | | | 1795 |

Men

| Food Category | Breakfast | Lunch | Dinner | Snack | Sub-total |
|---|---|---|---|---|---|
| Protein | 200 | 275 | 275 | 0 | 750 |
| Vegetable | 0 | 90 | 90 | 0 | 180 |
| Greens | 0 | 30 | 30 | 0 | 60 |
| Fruits | 100 | 100 | 0 | 0 | 200 |
| Grains | 150 | 150 | 150 | 0 | 360 |
| Oil & Seasoning | 60 | 60 | 60 | 0 | 180 |
| NTx (w/bev.) | 10 | 0 | 0 | 0 | 10 |
| Snack | 0 | 0 | 0 | 180 | 180 |
| Sub-total | 520 | 705 | 605 | 180 | |
| Total | | | | | 2010 |

Nutritional Therapeutics

AM Pill Pack:

NT5—1 capsules

NT12—2 capsules

NT19—1 capsules

NT20—1 capsule

Mid-Day Pill Pack:

NT5—1 capsules

NT12—2 capsules

NT19—1 capsules

NT21—1 capsule

PM Pill Pack:

NT5—1 capsules

NT18—1 capsules

NT22—1 capsules

NT23—1 capsule

NT19

This unique formula combines well-researched botanicals along with two up and coming herbs that more recently emerged in the literature, demonstrating impressive efficacy in the optimization of blood sugar control, resulting in a truly synergistic effect.

Serving Size: 4 capsules

(Amount per Serving % Daily Value)

Salacia (Salacia oblonga) 500 mg*(root and stems)

Fennugreek 500 mg*(Trigonella foenum-graecum) [standardized to contain 60% saponins]

American Ginseng 400 mg*(Panax quinquefolius) [standardized to contain 5% ginsenosides]

Gymnema 400 mg*(Gymnema sylvestre) [standardized to contain 25% gymnemic acid]

Banaba 400 mg*(Langerstroemia spp.) [standardized to contain 1% corosolic acid]

Kudzu 400 mg*(Pueraraia lobata) [standardized to contain 40% isoflavones]

Cinnamon (Cinnamomum spp.) 400 mg*[bark]

*Daily Value not established.

Other Ingredients: Microcrystalline cellulose, rice flour, magnesium stearate.

NT20

NT 20 provides an enhanced level of omega-3 fatty acids per serving. Lipase, a digestive aid, an enzyme needed for breaking down lipids (fats). Lipase is primarily produced in the pancreas but is also produced in the mouth and stomach.

Serving Size: 2 Soft Gels

(Amount Per Serving % Daily Value)

Omega 3 Fatty Acids 600 mg*[EPA (Eicosapentaenoic Acid)

DHA (Docosahexaenoic Acid) 400 mg*

Other Omega-3 Fatty Acids 200 mg*

*Percent Daily Values are based on a 2,000 calorie diet.

Other Ingredients: Bovine gelatin, glycerin, water, natural lemon flavor, lipase, annatto, and mixed tocopherols.

NT21

NT 21 Fuels the mitrochondria for fat burning. Without Carnitine fats cannot be burned for energy. Without energy, cells become weak or die. Because of this, optimizing Carnitine levels has been found to have dramatic benefits for low energy, obesity, chronic fatigue, immune deficiency conditions, and elevated cholesterol and triglycerides. Controlled trials have demonstrated that Carnitine increases weight loss by promoting optimal fat burning by the mitochondria. Carnitine also helps promote heart health, cholesterol lowering, sports endurance, and helps relieve chronic fatigue. Acetyl-L-Carnitine is also contained in NT21 and has unique brain health enhancing properties. NT21 is the only product of its kind combining both of these valuable forms of Carnitine into one capsule.

Serving Size: 2 Soft Gels

(Amount Per Serving % Daily Value)

L-Carnitine 400 mg*(from L-carnitine-tartrate)

Acetyl-L-Carnitine HCL 100 mg*

NT22

NT22 contains ubiquinol, the reduced, antioxidant form of CoQ10. Ubiquinol plays a primary role of decreasing oxidative damage caused by lipid peroxidation within mitochondria. According to research, plasma ubiquinol is decreased in participants with hyperlipidemia. NT22 may provide a strong initial stage defense against cellular oxidative damage and requires supplementing to maintain optimum health. CoQ10 is a natural chemical compound that humans make in our bodies and consume in our diets, primarily from oily fish, organ meats such as liver, and whole grains, that the body uses.

Ubiquinol 5 or 100 mg*(as Kaneka QH™ reduced form of CoQ10)
*Percent Daily Values are based on a 2,000 calorie diet.

NT23

Research of NT23 has proven it to have very potent antioxidant protection, estrogen protection, cardio-protection, cancer protection, viral protection and neuron protection. Studies have found that NT23 increases the production of a protein called SIRT1, and although it has not yet been confirmed in humans, in theory this action could increase human lifespan dramatically.

Serving Size: 1 Capsule

(Amount Per Serving % Daily Value)

Trans-Resveratrol 200 mg*(from 400 mg Polygonum cuspidatum, root)

Quercetin Dihydrate 200 mg*

Lecithin 100 mg*

All Protein is Not Created Equal

Protein, made up of amino acids, is essential to cell health, tissue growth and repair, and can help to curb appetite. There are a number of essential amino acids that the human body cannot manufacture—we must get them from an outside source. As the participant considers the many options available, there are a number of factors to keep in mind.

Animal Protein

Animal proteins are great for providing many necessary amino acids, but are not very “bio-available” or easily digested (many have only about 20% digestible protein). They do, however, increase the level of calorie burning, simply through the act of digesting them. In programs described herein, it can be advantageous to not to get all animal proteins from a single source, as they all have their downfalls—most beef is grown with antibiotics and has a higher fat content, many types of fish have high mercury content, and any chicken that isn't cage-free, free range organic is likely to contain arsenic.

Eggs are a great source of protein and vitamins. Many people still worry about cholesterol in the yolks and opt to toss them instead of eating them—that's not necessary. All of the vitamins and other nutrients are in the yolks, so while one shouldn't regularly load up on a half dozen yolks a day, one or two whole eggs a day will have no effect on one's cholesterol levels.

Whey Protein

Whey protein is a very bio-available and healthy protein, but careful attention should be paid to the specific type of whey protein obtained. Whey protein isolate is much better than whey protein concentrate, however it is typically more expensive. A whey protein product need only contain 1% isolate to be labeled a whey protein isolate. Also, some manufacturers also fortify their whey proteins with other amino acids—look for those high in branch chain amino acids (BCAAs), which are important for tissue growth and repair.

Soy Protein

Soy protein, while having many potential health benefits, is also a bit controversial. Soy contains phytoestrogens, which are “estrogen-like” compounds that bind to estrogen receptors in the body. Some research suggests that phytoestrogens can decrease testosterone levels in men and cause significant mood variability and gynocomastia (increased breast tissue). Other research suggests just the opposite—that these weak phytoestrogens prevent stronger estrogen from binding and therefore benefit men. Given that there is no consensus on the issue of phytoestrogens and men's health, a program as described herein includes consuming soy proteins in moderation. Limiting high quality soy protein to 15-20% of one's total protein intake should keep the participant well below levels that could create negative health effects, while still allowing the participant to reap the reported positive effects of soy, such as improvements in the markers of cardiac health.

Plant Protein

Plant proteins such as beans, rice, and pea proteins generally don't have a full essential amino acid complement, and need to be combined. Properly processed rice protein, combined with yellow pea protein, is extremely bio-available and has an excellent amino acid profile. Unfortunately, it's not always the easiest to find. Ultimately, participants advantageously try to limit animal proteins to those that are organic, and try to get protein from multiple sources to promote muscle growth, tissue healing, and good health in general.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

1. A therapeutic method for a human participant, the method comprising:
(1) detoxifying the participant while consuming by the participant a diet having a calorie content sufficiently low to induce an anti-inflammatory effect in the participant, said detoxifying being at a level higher than normally occurs for said calorie content consumed by the participant;
(2) after (1), consuming by the participant a low-calorie diet with a higher calorie content than in step (1);
(3) after (2), consuming by the participant a medium-calorie diet with a higher calorie content than said low-calorie diet in step (2); and
(4) after (3), consuming by the participant a diet with a higher calorie content than the medium-calorie diet.

2. A method according to claim 1, wherein detoxifying comprises:
building a nutrient base in the participant;
restoring the participant's digestive health;
reducing inflammation in the participant; or
combinations thereof.

3. A method according to claim 1, wherein consuming by the participant a low-calorie diet comprises:
improving the participant's health markers;
resetting the participant's metabolism;
resetting the participant's hunger; or
combinations thereof.

4. A method according to claim 1, wherein consuming by the participant a medium-calorie diet further comprises:
providing to the participant nutrition education;
stabilizing the participant's weight;
integrating an exercise program for the participant; or
combinations thereof.

5. A method according to claim 1, wherein consuming by the participant a diet with a higher calorie content than the medium-calorie diet further comprises controlling the participant's hunger, including:
stabilizing the patient's blood sugar level;
reducing inflammation in the participant;
supporting the participant's metabolism; or
combinations thereof.

6. A method according to claim 2, wherein reducing inflammation in the participant comprises:
consuming, by the participant, a therapeutically effective amount of an antioxidant, an essential fatty acids, a natural COX-2 inhibitor, or combination thereof;
eliminating dietary allergens, irritants, chemicals, toxins, or combinations thereof, during said detoxifying; or
both.

7. A method according to claim 6, wherein said essential fatty acids comprise EPA, DHA, or both.

8. A method according to claim 1, further comprising:
lipid-loading during said detoxifying the participant.

9. A method according to claim 1, wherein detoxifying the participant further comprises:
consuming, by the participant, nutritional supplements to support liver function, in an amount greater than present in said diet having a calorie content sufficiently low to induce an anti-inflammatory effect.
